(19) United States
(12) Patent Application Publication
(10) Pub. No.: US 2003/0228317 A1
(43) Pub. Date: Dec. 11, 2003

(54) Gene BRCC-1 and diagnostic and therapeutic uses thereof

(76) Inventors: Prafulla Gokhale, Oak Hill, VA (US); Anatoly Dritschilo, Bethesda, MD (US); Aquilur Rahman, Potomac, MD (US); Imran Ahmad, Wadsworth, IL (US); Usha Kasid, Rockville, MD (US)

Correspondence Address: LEYDIG VOIT & MAYER, LTD, TWO PRUDENTIAL PLAZA, SUITE 4900, 180 NORTH STETSON AVENUE, CHICAGO, IL, 60601-6780, US

(21) Appl. No.: 10/443,273

(22) Filed: May 22, 2003

Related U.S. Application Data
(60) Provisional application No. 60/382,031, filed on May 22, 2002.

(51) Int. Cl. A61K 39/395; C12Q 1/68; C07H 21/04; C12N 9/64; C12P 21/02; C12N 5/06

(52) U.S. Cl. 424/146.1; 435/6; 435/69.1; 435/226; 435/320.1; 435/325; 536/23.2

(57) ABSTRACT

A gene that is a modulator of tumor growth and metastasis in certain cancer types is provided. This gene and corresponding polypeptide have diagnostic and therapeutic application for detecting and treating cancers that involve expression of BRCC-1 such as breast cancer and lung cancer.

### CROSS-REFERENCE TO ELATED APPLICATIONS

This application claims priority to U.S. Provisional Patent Application No. 60/382,031, filed May 22, 2002.

### FIELD OF THE INVENTION

The present invention relates to a gene that encodes a polypeptide that modulates apoptosis. This polypeptide is a useful target for identifying compounds that inhibit cancer progression by modulating apoptosis. Also, this polypeptide is useful as a diagnostic target for detecting cancers wherein this polypeptide is differentially expressed, e.g., breast cancer, lung cancer, etc.

### BACKGROUND OF THE INVENTION

Neoplasia is the relatively autonomous proliferation of cells, whereby cells partially or totally escape physiological control mechanisms that ordinarily constrain cell proliferation and regulate cell differentiation. The proliferation of normal cells is believed regulated by growth-promoting proto-oncogenes counterbalanced by growth-constraining tumor-suppressor genes. Mutations that potentiate the activities of proto-oncogenes can create the oncogenes that force the deregulated growth of neoplastic cells. Conversely, genetic lesions that inactivate tumor suppressor genes, generally through mutation(s) that lead to a cell being homozygous for the inactivated tumor suppressor allele, can liberate the cell from the normal replicative constraints imposed by these genes. Often, an inactivated tumor suppressor gene in combination with the formation of an activated oncogene (i.e., a proto-oncogene containing an activating structural or regulatory mutation) can yield a neoplastic cell capable of essentially unconstrained growth (i.e., a transformed cell).

Many pathological conditions result, at least in part, from aberrant control of cell proliferation, differentiation, and/or apoptosis. For example, neoplasia is characterized by a clonally derived cell population which has a diminished capacity for responding to normal cell proliferation control signals. Oncogenic transformation of cells leads to a number of changes in cellular metabolism, physiology, and morphology. One characteristic alteration of oncogenically transformed cells is a loss of responsiveness to constraints on cell proliferation and differentiation normally imposed by the appropriate expression of cell growth regulatory genes.

The precise molecular pathways and secondary changes leading to malignant transformation for many cell types are not entirely clear. Oncogenic transformation of cells leads to a number of changes in cellular metabolism, physiology, and morphology. One characteristic alteration of oncogenically transformed cells is a loss of responsiveness to constraints on cell proliferation and differentiation normally imposed through one or more signaling pathway(s) which comprise proteins encoded by proto-oncogenes. For example, proteins encoded by ras genes serve as essential transducers of diverse physiological signals, and mutationally altered ras gene products are important contributors to the neoplastic phenotype. The 21 kilodalton protein encoded by the rasH gene, referred to as p21ras, is involved in the signal transduction of various factors controlling cell proliferation, differentiation, and oncogenesis.

Investigations have revealed several proteins that function upstream and downstream of p21ras in signaling pathways. In particular, a protein encoded by the raf-1 proto-oncogene functions downstream of p21ras and is implicated in several other signaling pathways, including T cell receptor stimulation (Siegel et al. (1993) J. Immunol. 151: 4116; Wotton etal. (1993) J.Biol. Chem. 268: 17975; Prasad K V and Rudd C E (1992) Mol. Cell. Biol. 12: 5260), muscarinic m2 receptor stimulation (Winitz et al. (1993) J. Biol. Chem. 268: 19196), TPA/protein kinase C-stimulation and TNF-.alpha. receptor stimulation (Finco et al. (1993) J. Biol. Chem. 268: 17676; Sozeri et al. (1992) Oncogene 7: 2259), IL-2 receptor stimulation (Turner et al. (1993) Proc. Natl. Acad. Sci. (USA). 90: 5544), nerve growth factor receptor stimulation (Ohmichi et al. (1992) J. Biol. Chem. 267: 14604), erythropoietin-mediated proliferation (Carroll et al. (1991) J. Biol. Chem. 266: 14964), and various mitogenic signaling pathways such as EGF and PDGF receptor stimulation (Kizaka-Kondoh et al. (1992) Mol. Cell. Biol. 12: 5078; Baccarini et al. (1991) J. Biol. Chem. 266: 10941) and\hat{ }insulin stimulation (Lee et al. (1991) J. Biol. Chem. 266: 10351).

The raf-1 protein is a serine/threonine kinase that is structurally related to the protein kinase C (PKC) family, and is essential in cell growth and differentiation. A variety of upstream signaling pathways lead to raf-1, which exhibits an enhanced kinase activity when activated via an upstream signaling pathway. The exact biochemical alterations that define activation of raf-1 have not been rigorously defined. The raf-1 protein and p21ras have been found to physically associate with each other via the amino-terminal portion of raf-1, but this association is not itself sufficient to activate raf-1 kinase activity (Fabian et al. (1993) Mol. Cell. Biol. 13: 7170; Traverse et al. (1993) Oncogene 8: 3175; Koide et al. (1993) Proc. Natl. Acad. Sci. (USA) 90: 8683; Warne et al. (1993) Nature 364: 352; Zhang et al. (1993) Nature 364: 308). The portions of raf-1 which confer binding specificity towards other proteins remains to be elucidated, as does the molecular identification of such other raf-1 binding proteins. Recent studies of growth factor signal transduction pathways have shown that raf-1 functions downstream of several activated tyrosine kinases as well as p21ras and functions upstream of mitogen-activated protein kinase (MAP kinase). Thus, in addition to p21ras, a variety of oncogene proteins and receptors having tyrosine kinase activity can activate the kinase activity of raf-1 towards various substrates, which then modulate downstream signaling (Gardner et al. (1993) J. Biol. Chem. 268: 17896). The raf-1 protein becomes phosphorylated on tyrosine residue(s) in response to upstream signals, such as by growth factor stimulation, and this phosphorylation is involved in the activation of raf-1 kinase activity (Fabian et al. (1993) op.cit.; Morrison et al. (1993) J. Biol. Chem. 268: 17309). Inhibiting raf-1 function blocks mitogen-activated protein kinase activation by growth factors and p21ras (Schaap et al. (1993) J. Biol. Chem. 268; 20232; Samuels et al. (1993) Mol. Cell. Biol. 13:6241).

Once activated, raf-1 manifests a serine/threonine kinase activity which acts on a variety of polypeptide substrates that comprise one or more downstream signaling pathways. Recently, oncogenically activated raf-1 has been demonstrated to activate MAP kinase, which leads to the phosphorylation and activation of various MAP kinases, such as the extracellular signal-regulated kinases ERK1 and ERK2 (Howe et al. (1992) Cell 71: 335; Kyriakis et al. (1992) Nature 358: 417). MAP kinases appears to be a central component of many different signal transduction pathways, and activation of MAP kinases has been shown to direct phosphorylation of transcription factors, such as c-myc, c-jun, and p62TcF (Gille et al. (1992) Nature 358: 414; Alvarez et al. (1991) J. Biol. Chem. 266; 15277; Pulverer et al. (1991) Nature 353: 670; Seth et al. (1991) J. Biol. Chem. 266: 23521) and activation of other kinases, such as p90rsk (Sturgill et al. (1988) Nature 334; 715; Chung et al (1991) Proc. Natl. Acad. Sci. (USA) 88: 4981) and MAPKAP kinase 2 (Stokoe et al. (1992) EMBO J. 11: 3985). Macdonald et al. (1993) Mol. Cell. Biol. 13:6615 have shown that MEK (MAP/ERK kinase) is a direct phosphorylation substrate of raf-1, and that phosphorylation of MEK by raf-1 is sufficient for MEK activation.

Since many of the signaling pathway(s) which are mediated by activation of the kinase activity of raf-1 are involved in control of cell proliferation and oncogenic transformation, it would be desirable to identify other physiologically relevant proteins to which raf-1 binds. Moreover, it would be desirable to have agents which modulate the activity of raf-1, such as agents which interfere with the binding of raf-1 to other proteins, particularly inhibiting binding of raf-1 to proteins involved in the control of the cell cycle and/or cell differentiation. Such raf-1 blocking agents can be administered to a human or veterinary patient in a pharmaceutically acceptable form and in a therapeutically effective dosage for prophylaxis and therapy of diseases, including neoplasia, hyperplasia, and other pathological conditions related to elevated or prolonged raf-1 activity. Preferably, such raf-1 blocking agents will be small molecules or peptidomimetics which have advantageous pharmacokinetic properties, such as a desirable half-life, low toxicity, ready deliverability to various tissues and organs, facile passage across cell membranes to gain access to intracellular raf-1, and the like. Advantageously, such raf-1 blocking agents will also find use as commercial reagents, for example, to modulate a cultured cell phenotype for laboratory purposes and/or for bioprocess control (e.g., to prevent excessive cell proliferation in a bioreactor culture), and the like.

Despite progress in developing a more defined model of the molecular mechanisms underlying the transformed phenotype and neoplasia, few significant therapeutic methods applicable to treating cancer beyond conventional chemotherapy have resulted. The observation that aberrant raf-1 function is frequently correlated with neoplasia supports a model wherein raf-1 protein is involved in control of cell proliferation, and may be involved in one or more signaling pathways that transduce growth regulatory signals. If such a model were correct, raf-1 and biological macromolecules (i.e., proteins) that specifically interact with raf-1 would be candidate targets for therapeutic manipulation. For example and not limitation, if a hypothetical protein X bound to raf-1 forming a complex and thereby stimulated (or alternatively, inhibited) neoplastic growth of cells, agents that would selectively inhibit (or alternatively, augment) formation of the protein X: raf-1 complex or otherwise modulate raf-1 activity may be candidate antineoplastic agents.

The identification of proteins that interact with raf-1 protein provide a basis for screening assays for identifying agents that specifically interfere with the intermolecular association between raf-1 protein and such interacting proteins. These screening assays can be used to identify candidate raf-1 modulating agents that can serve as candidate therapeutic agents. Such raf-1 modulating agents can provide novel chemotherapeutic agents for treatment of neoplasia, cell proliferative conditions, arthritis, inflammation, autoimmune diseases, and the like. The present invention fulfills these and other needs.

Based on the foregoing, it is clear that a need exists for agents that inhibit raf-1 (e.g., inhibit binding of raf-1 to other proteins involved in signal transduction and/or growth control) and which are pharmaceutically acceptable for use in humans and veterinary patients to treat diseases characterized by undesired raf-1 activity, such as cancer, hyperplasia, and the like. Thus, it is an object of the invention herein to provide such raf-1 blocking agents, compositions of such agents, methods of treating diseases resulting from excessive raf-1 activation (e.g., neoplasia, hyperplasia), and novel pharmaceutical compositions comprising a raf-1 inhibitory agent in combination with one or more additional antineoplastic agents.

Alterations in the cellular genes which directly or indirectly control cell growth and differentiation are considered to be the main cause of cancer. The raf gene family includes three highly conserved genes termed A-, B- and c-raf (also called raf-1). Raf genes encode protein kinases that are thought to play important regulatory roles in signal transduction processes that regulate cell proliferation. Expression of the c-raf protein is believed to play a role in abnormal cell proliferation since it has been reported that 60% of all lung carcinoma cell lines express unusually high levels of c-raf mRNA and protein. Rapp et al., The Oncogene Handbook, E. P. Reddy, A. M Skalka and T. Curran, eds., Elsevier Science Publishers, New York, 1988, pp. 213-253.

Malignant tumors develop through a series of stepwise, progressive changes that lead to the loss of growth control characteristic of cancer cells, i.e., continuous unregulated proliferation, the ability to invade surrounding tissues, and the ability to metastasize to different organ sites. Carefully controlled in vitro studies have helped define the factors that characterize the growth of normal and neoplastic cells and have led to the identification of specific proteins that control cell growth and differentiation.

As discussed above, the raf genes are members of a gene family which encode related proteins termed A-, B- and c-raf. Raf genes code for highly conserved serine-threonine-specific protein kinases. These enzymes are differentially expressed; c-raf, the most thoroughly characterized, is expressed in all organs and in all cell lines that have been examined. A- and B-raf are expressed in urogenital and brain tissues, respectively, c-raf protein kinase activity and subcellular distribution are regulated by mitogens via phosphorylation. Various growth factors, including epidermal growth factor, acidic fibroblast growth factor, platelet-derived growth factor, insulin, granulocyte-macrophage colony-stimulating factor, interleukin-2, interleukin-3 and erythropoietin, have been shown to induce phosphorylation of c-raf. Thus, c-raf is believed to play a fundamental role in the normal cellular signal transduction pathway, coupling a multitude of growth factors to their net effect, cellular proliferation.

Certain abnormal proliferative conditions are believed to be associated with raf expression and are, therefore, believed to be responsive to inhibition of raf expression. Abnormally high levels of expression of the raf protein are also implicated in transformation and abnormal cell proliferation. These abnormal proliferative conditions are also believed to be responsive to inhibition of raf expression. Examples of abnormal proliferative conditions are hyperproliferative disorders such as cancers, tumors, hyperplasias, pulmonary fibrosis, angiogenesis, psoriasis, atherosclerosis and smooth muscle cell proliferation in the blood vessels, such as stenosis or restenosis following angioplasty. The cellular signaling pathway of which raf is a part has also been implicated in inflammatory disorders characterized by T-cell proliferation (T-cell activation and growth), such as tissue graft rejection, endotoxin shock, and glomerular nephritis, for example.

Oligonucleotides have been employed as therapeutic moieties in the treatment of disease states in animals and man. For example, workers in the field have now identified antisense, triplex and other oligonucleotide compositions which are capable of modulating expression of genes implicated in viral, fungal and metabolic diseases. Antisense oligonucleotide inhibition of gene expression has proven to be a useful tool in understanding the roles of raf genes. An antisense oligonucleotide complementary to the first six codons of human c-raf has been used to demonstrate that the mitogenic response of T cells to interleukin-2 (IL-2) requires c-raf. Cells treated with the oligonucleotide showed a near-total loss of c-raf protein and a substantial reduction in proliferative response to IL-2. Riedel et al., Eur. J. Immunol. 1993, 23, 3146-3150. Rapp et al. have disclosed expression vectors containing a raf gene in an antisense orientation downstream of a promoter, and methods of inhibiting raf expression by expressing an antisense Raf gene or a mutated Raf gene in a cell. Wo application 93/04170. An antisense oligodeoxyribonucleotide complementary to codons 1-6 of murine c-Raf has been used to abolish insulin stimulation of DNA synthesis in the rat hepatoma cell line H4IIE. Tornkvist et al., J. Biol. Chem. 1994, 269, 13919-13921. WO Application 93/06248 discloses methods for identifying an individual at increased risk of developing cancer and for determining a prognosis and proper treatment of patients afflicted with cancer comprising amplifying a region of the c-raf gene and analyzing it for evidence of mutation. Denner et al. disclose antisense polynucleotides hybridizing to the gene for raf, and processes using them. Wo 94/15645. Oligonucleotides hybridizing to human and rat raf sequences are disclosed. Iversen et al. disclose heterotypic antisense Oligonucleotides complementary to raf which are able to kill ras-activated cancer cells, and methods of killing raf-activated cancer cells. Numerous oligonucleotide sequences are disclosed, none of which are actually antisense oligonucleotide sequences.

U.S. Pat. No. 5,919,773, to Monia et al discloses that elimination or reduction of raf gene expression can halt or reverse abnormal cell proliferation. The Moinia et al patent discloses Oligonucleotides targeted to nucleic acids encoding raf. This relationship between an oligonucleotide and its complementary nucleic acid target to which it hybridizes is commonly referred to as “antisense.”

It is noted however, that raf-1 involvement marks only a component in a complex growth and cell survival/death pathway, the identification of other components of which may allow more selective, more specific and/or more efficacious targeting of such components. Identification of one or more genes associated with such components would highly beneficial.

### OBJECTS AND SUMMARY OF THE INVENTION

In one aspect, the invention provides a novel gene that encodes a polypeptide which is a component of cell survival/death pathway which is other than the raf-1 component. In another aspect, the invention provides a BRCC-1 nucleic acid sequence having SEQ ID NO 2. In another aspect, the invention provides a BRCC-1 nucleic acid sequence encoding the BRCC-1 protein and having SEQ ID NO 3, or a homolog or analog thereof, that encodes a polypeptide having at least 90% sequence identity to said polypeptide, or a fragment thereof that encodes a polypeptide that modulates apoptosis. In another aspect, the invention provides a BRCC-1 polypeptide that modulates apoptosis comprising the amino acid sequence contained in SEQ ID NO 3 or a fragment thereof which is at least 50 amino acids in length or an analog or homolog having at least 90% sequence identity to said polypeptide which modulates apoptosis.

In another aspect, the invention provides an antibody that specifically binds BRCC-1 polypeptide. In another aspect, the invention provides a method for identifying compounds that modulate apoptosis by screening for compounds that specifically bind BRCC-1 polypeptide.

In another aspect, the invention provides a method for detecting or evaluating the prognosis of a cancer characterized by differential expression of BRCC-1 by detecting expression of BRCC-1 in an analyte obtained from a patient tested for cancer and correlating the level of expression to a positive or negative diagnosis for cancer.

In another aspect, the invention provides a method of treating or preventing a cancer characterized by differential expression of BRCC-1 comprising administering a compound that modulates BRCC-1 gene expression and/or activity of BRCC-1 polypeptide. In another aspect, the invention provides a method for treating cancer comprising administering at least one antisense oligonucleotide or ribozyme that inhibits BRCC-1 expression, thereby inhibiting cancer cell proliferation and/or metastatic potential. In another aspect, the invention provides a method for treating cancer comprising administering BRCC-1 cDNA that leads to overexpression of BRCC-1, thereby inhibiting cancer cell proliferation and/or metastatic potential.

In another aspect, the invention provides a pharmaceutical composition for the treatment of cancer that comprises an antagonist of BRCC-1 expression and/or activity and a pharmaceutically acceptable carrier. It is still another object of the invention to provide a pharmaceutical composition for the treatment of cancer that comprises an agent causing overexpression of BRCC-1 and/or its activity and a pharmaceutically acceptable carrier. Preferably, such compositions will comprise liposomal formulations.

In another aspect, the invention provides diagnostic compositions for detection of cancer that comprise an oligonucleotide that specifically binds BRCC-1 DNA or an antibody that specifically binds the BRCC-1 polypeptide, attached directly or indirectly to a label, and a diagnostically acceptable carrier.

In another aspect, the invention provides methods for inhibiting tumor growth and/or metastasis by administration of a molecule that antagonizes the expression and/or activity of BRCC-1.

In another aspect, the invention provides liposomal formulations for antisense therapy that inhibit tumor growth and/or metastasis which comprise antisense oligonucleotides specific to BRCC-1, optionally in association with cytotoxic moieties such as radionuclides,

### DETAILED DESCRIPTION OF THE FIGURES

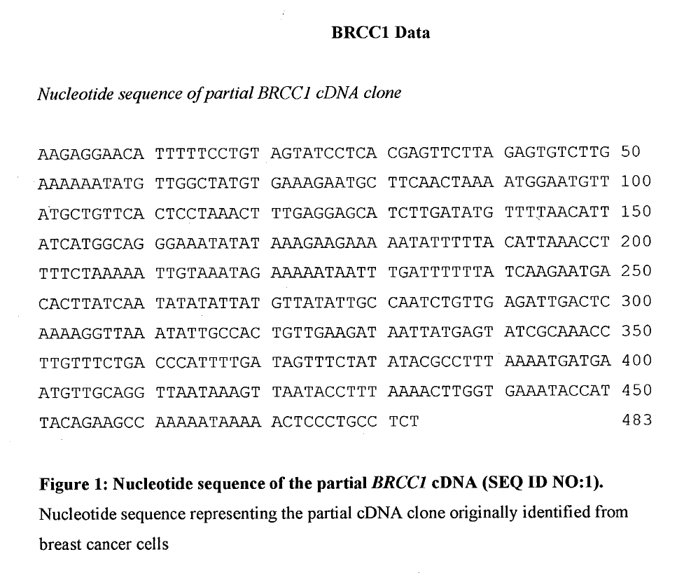

FIG. 1: Nucleotide sequence of the partial BRCC1 cDNA. Nucleotide sequence representing the partial cDNA clone originally identified from breast cancer cells (GenBank Accession Number: AF220060, date of submission Dec. 29, 1999).

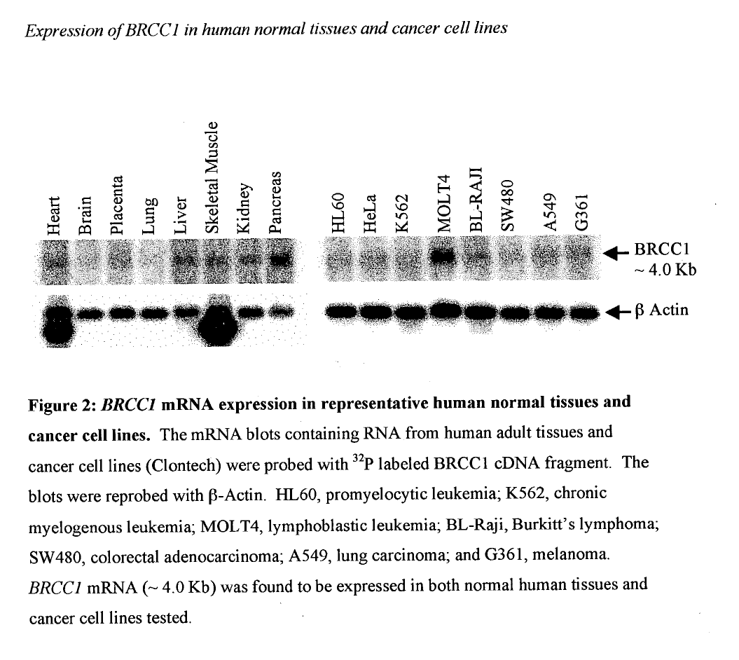

FIG. 2: BRCC 1 mRNA expression in representative human normal tissues and cancer cell lines. The mRNA blots containing RNA from human adult tissues and cancer cell lines (Clontech) were probed with 32P labeled BRCC1 cDNA fragment. The blots were reprobed with p-Actin. HL60, promyelocytic leukemia; K562, chronic myelogenous leukemia; MOLT4, lymphoblastic leukemia; BL-Raji, Burkitt's lymphoma; SW480, colorectal adenocarcinoma; A549, lung carcinoma; and G361, melanoma. BRCC1 mRNA (˜4.0 Kb) was found to be expressed in both normal human tissues and cancer cell lines tested.

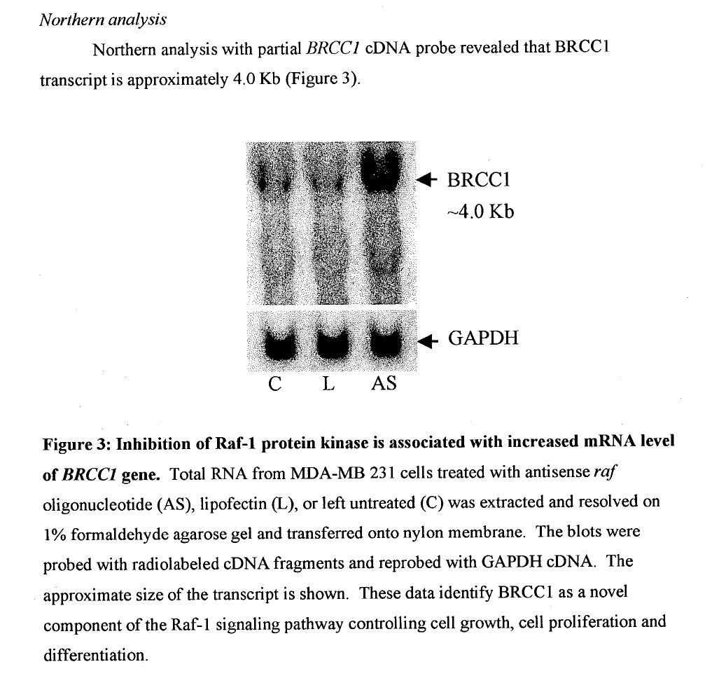

FIG. 3: Inhibition of Raf-I protein kinase is associated with increased mRNA level of BRCC 1 gene. Total RNA from MDA-MB 231 cells treated with antisense raf oligonucleotide (AS), lipofectin (L), or left untreated (C) was extracted and resolved on 1% formaldehyde agarose gel and transferred onto nylon membrane. The blots were probed with radiolabeled cDNA fragments and reprobed with GAPDH cDNA. The approximate size of the transcript is shown. These data identify BRCC1 as a novel component of the Raf-1 signaling pathway controlling cell growth, cell proliferation and differentiation.

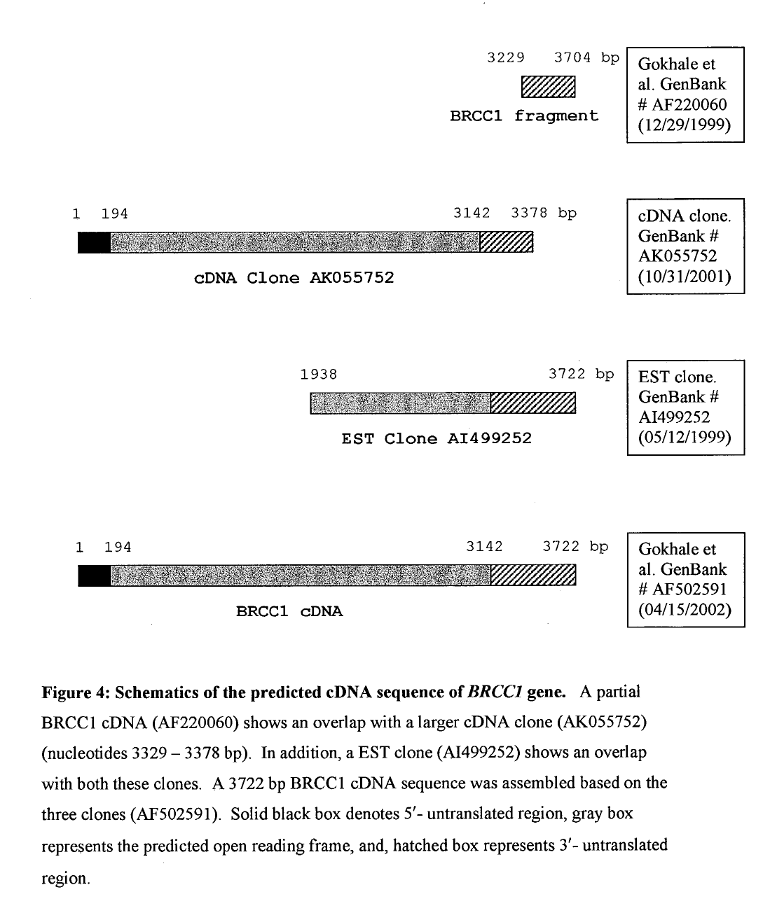

FIG. 4: Schematics of the predicted cDNA sequence of BRCC1 gene. A partial BRCC1 cDNA (AF220060) shows an overlap with a larger cDNA clone (AK055752) (nucleotides 3329 -3378 bp). In addition, a EST clone (AI499252) shows an overlap with both these clones. A 3722 bp BRCC1 cDNA sequence was assembled based on the three clones (AF502591). Solid black box denotes 5′- untranslated region, gray box represents the predicted open reading frame, and, hatched box represents 3′-untranslated region.

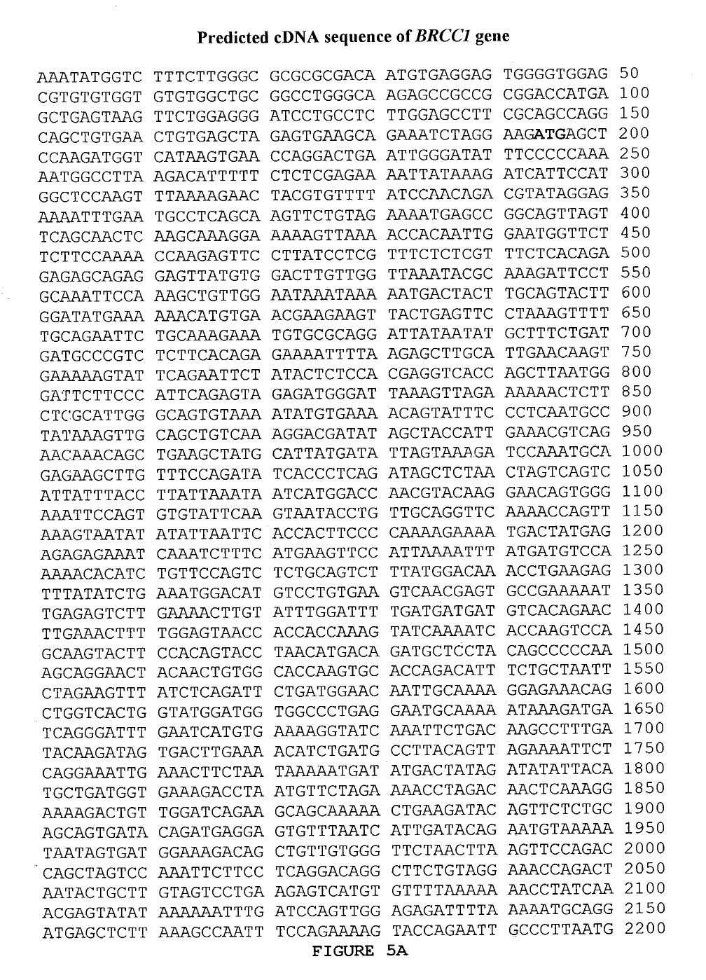

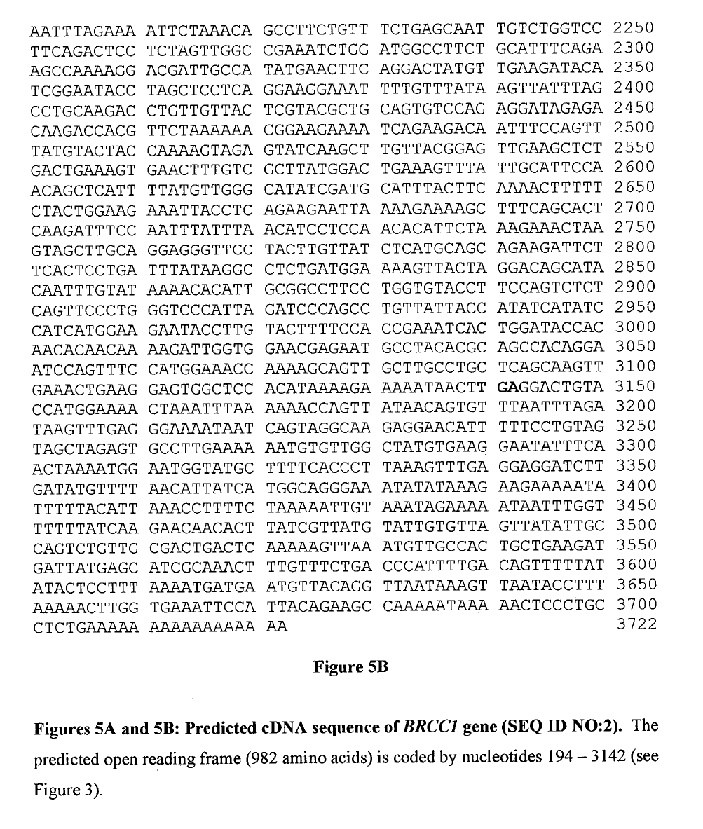

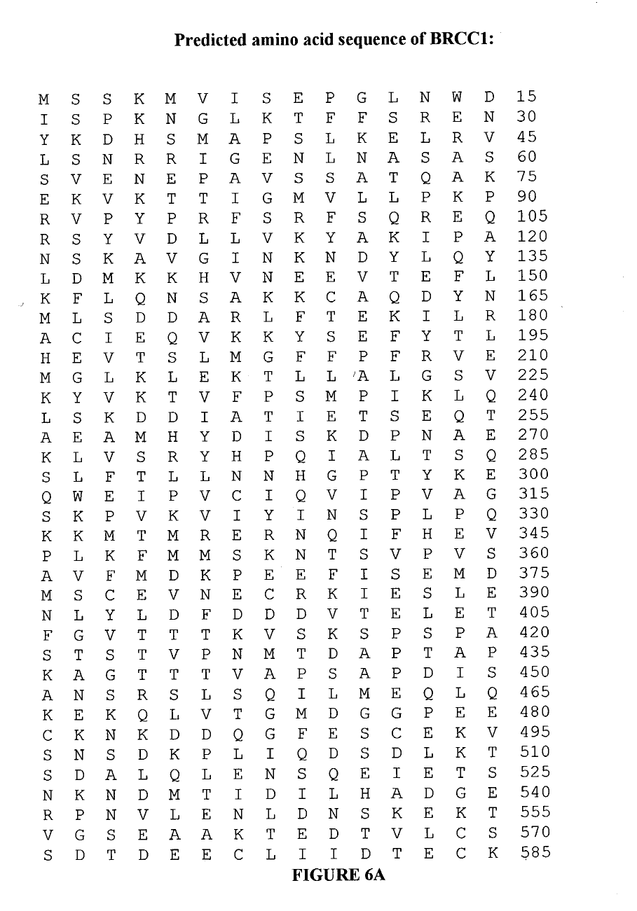

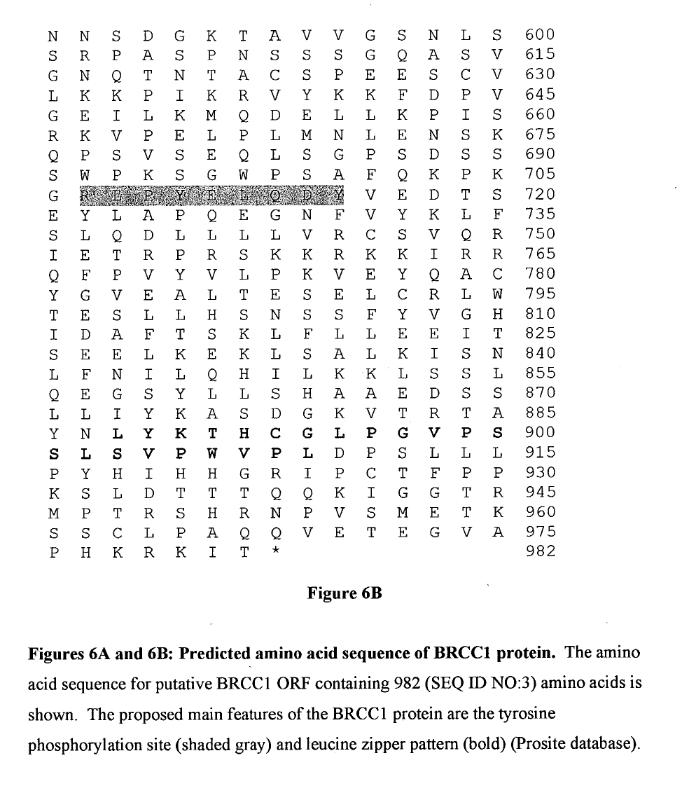

FIG. 5: Predicted cDNA sequence of BRCC1 gene (GenBank Accession # AF502591, Date of submission Apr. 15, 2002). The predicted open reading frame (982 amino acids) is coded by nucleotides 194 -3142 (see FIG. 6).

FIG. 6: Predicted amino acid sequence of BRCC1 protein. The amino acid sequence for putative BRCC1 ORF containing 982 amino acids is shown (AF502591, submission date Apr. 15, 2002). The proposed main features of the BRCC1 protein are the tyrosine phosphorylation site (shaded gray) and leucine zipper pattern (bold) (Prosite database).

### DETAILED DESCRIPTION OF THE INVENTION

The molecular genetic factors that negate cell death and contribute to tumor growth and metastasis can be attractive targets for therapeutic intervention. In a search for such genes, the present inventors have identified a full length cDNA encoding a gene which is hereby named as BRCC-1 that is a modulator of apoptosis.

The expression of the gene BRCC-1 is differentially expressed in human breast cancer cells treated with an antisense raf oligonucleotide (AS-raf-ODN) (FIG. 3). AS-raf-ODN causes programmed cell death in cancer cells by decreasing the amount of a proliferation and survival-promoting protein Raf-1. Thus, BRCC-1 is a component of growth and cell survival/death pathway in cancer cells and is regulated by raf-1 protein. In linking the expression of BRCC-1 to the modulation of Raf-1, the present inventors have shown that BRCC-1 is up-regulated by Raf-1 inhibition and therefore this newly discovered gene is a component of the cell survival/cell death pathway. More particularly, the present invention is based, at least in part, on the discovery that the BRCC-1 gene plays a role down stream of Raf-1.

Other aspects of the present invention are based on the discovery that the manipulation of the level of BRCC-1 in cancer cells provides therapeutic advantages. For example, increasing the amount of BRCC-1 induces many of the in vivo effects of antisense raf oligonucleotide such as tumor growth arrest, tumor regression, tumor cell death and/or potentiate radiation/drug-induced cytotoxicity. In addition, being a target potentially downstream of Raf-1, it is anticipated that greater specificity is obtained by targeting the action of BRCC-1 as compared with Raf-1.

The present invention relates to a novel gene, BRCC-1, that modulates apoptosis, the corresponding polypeptide, and application thereof in diagnostic and therapeutic methods. Particularly, the invention provides a novel target for identifying compounds that promote or inhibit apoptosis of cancer cells, especially breast and lung cancer.

As noted, the invention is broadly directed to a novel gene referred to as BRCC-1. Reference to BRCC-1 herein is intended to be construed to include BRCC-1 proteins of any origin which are substantially homologous to and which are biologically equivalent to the BRCC-1 characterized and described herein. Such substantially homologous BRCC-1 may be native to any tissue or species and, similarly, biological activity can be characterized in any of a number of biological assay systems.

The term “biologically equivalent” is intended to mean that the compositions of the present invention are capable of demonstrating some or all of the same biological properties in a similar fashion, not necessarily to the same degree as the BRCC-1 isolated as described herein or recombinantly produced human BRCC-1 of the invention.

By “substantially homologous” it is meant that the degree of homology of human BRCC-1 from any species is greater than that between BRCC-1 and any previously reported apoptopic modulating gene.

Sequence identity or percent identity is intended to mean the percentage of same residues between two sequences, wherein the two sequences are aligned using the Clustal method (Higgins et al, Cabios 8:189-191, 1992) of multiple sequence alignment in the Lasergene biocomputing software (DNASTAR, INC, Madison, Wis.). In this method, multiple alignments are carried out in a progressive manner, in which larger and larger alignment groups are assembled using similarity scores calculated from a series of pairwise alignments. Optimal sequence alignments are obtained by finding the maximum alignment score, which is the average of all scores between the separate residues in the alignment, determined from a residue weight table representing the probability of a given amino acid change occurring in two related proteins over a given evolutionary interval. Penalties for opening and lengthening gaps in the alignment contribute to the score. The default parameters used with this program are as follows: gap penalty for multiple alignment=IO; gap length penalty for multiple alignment=10; k-tuple value in pairwise alignment=1; gap penalty in pairwise alignment=3; window value in pairwise alignment=5; diagonals saved in pairwise alignmentz=5. The residue weight table used for the alignment program is PAM250 (Dayhoffet al., in Atlas of Protein Sequence and Structure, Dayhoff, Ed., NDRF, Washington, Vol. 5, suppl. 3, p. 345, 1978).

Percent conservation is calculated from the above alignment by adding the percentage of identical residues to the percentage of positions at which the two residues represent a conservative substitution (defined as having a log odds value of greater than or equal to 0.3 in the PAM250 residue weight table). Conservation is referenced to human BRCC-1 when determining percent conservation with non-human BRCC-1, and referenced to BRCC-1 when determining percent conservation with non-BRCC-1 proteins. Conservative amino acid changes satisfying this requirement are: R-K; E-D, Y-F, L-M; V-1, Q-H.

The invention provides polypeptide fragments of the disclosed proteins. Polypeptide fragments of the invention can comprise or consist essentially of at least 8, 10, 12, 15, 18, 19, 20, 25, 50, 75, 100, or 200 contiguous amino acids of the amino acid sequence contained in FIG. 6 (SEQ ID NO 3). Also included are all intermediate length fragments in this range, such as 51, 52, 53, etc.; 70, 71, 72, etc.; and 100, 101, 102, etc., which are exemplary only and not limiting.

Variants of the BRCC-1 polypeptide disclosed herein can also occur. Variants can be naturally or non-naturally occurring. Naturally occurring variants are found in humans or other species and comprise amino acid sequences which are substantially identical to the amino acid sequence shown in FIG. 6 (SEQ ID NO 3). Species homologs of the protein can be obtained using subgenomic polynucleotides of the invention, as described below, to make suitable probes or primers to screening cDNA expression libraries from other species, such as mice, monkeys, yeast, or bacteria, identifying cDNAs which encode homologs of the protein, and expressing the cDNAs as is known in the art.

Non-naturally occurring variants which retain substantially the same biological activities as naturally occurring protein variants are also included here. Preferably, naturally or non-naturally occurring variants have amino acid sequences which are at least 85%, 90%, or 95% identical to the amino acid sequence shown in FIG. 6 (SEQ ID NO 3). More preferably, the molecules are at least 96%, 97%, 98% or 99% identical. Percent identity is determined using any method known in the art. A non-limiting example is the Smith-Waterman homology search algorithm using an affine gap search with a gap open penalty of 12 and a gap extension penalty of 1. The Smith-Waterman homology search algorithm is taught in Smith and Waterman, Adv. Appl. Math. (1981) 2:482-489.

Guidance in determining which amino acid residues can be substituted, inserted, or deleted without abolishing biological or immunological activity can be found using computer programs well known in the art, such as DNASTAR software. Preferably, amino acid changes in protein variants are conservative amino acid changes, i.e., substitutions of similarly charged or uncharged amino acids. A conservative amino acid change involves substitution of one of a family of amino acids which are related in their side chains. Naturally occurring amino acids are generally divided into four families: acidic (aspartate, glutamate), basic (lysine, arginine, histidine), non-polar (alanine, valine, leucine, isoleucine, proline, phenylalanine, methionine, tryptophan), and uncharged polar (glycine, asparagine, glutamine, cystine, serine, threonine, tyrosine) amino acids. Phenylalanine, tryptophan, and tyrosine are sometimes classified jointly as aromatic amino acids.

A subset of mutants, called muteins, is a group of polypeptides in which neutral amino acids, such as serines, are substituted for cysteine residues which do not participate in disulfide bonds. These mutants may be stable over a broader temperature range than native secreted proteins. See Mark et al., U.S. Pat. No. 4,959,314.

It is reasonable to expect that an isolated replacement of a leucine with an isoleucine or valine, an aspartate with a glutamate, a threonine with a serine, or a similar replacement of an amino acid with a structurally related amino acid will not have a major effect on the biological properties of the resulting secreted protein or polypeptide variant. Properties and functions of BRCC-1 or polypeptide variants are of the same type as a protein comprising the amino acid sequence encoded by the nucleotide sequence shown in FIG. 5 (SEQ ID NO 2), although the properties and functions of variants can differ in degree.

BRCC-1 protein variants include glycosylated forms, aggregative conjugates with other molecules, and covalent conjugates with unrelated chemical moieties. BRCC-1 protein variants also include allelic variants, species variants, and muteins. Truncations or deletions of regions which do not affect the differential expression of the BRCC-1 protein gene are also variants. Covalent variants can be prepared by linking functionalities to groups which are found in the amino acid chain or at the N- or C-terminal residue, as is known in the art.

It will be recognized in the art that some amino acid sequence of the BRCC-1 protein of the invention can be varied without significant effect on the structure or function of the protein. If such differences in sequence are contemplated, it should be remembered that there are critical areas on the protein which determine activity. In general, it is possible to replace residues that form the tertiary structure, provided that residues performing a similar function are used. In other instances, the type of residue may be completely unimportant if the alteration occurs at a non-critical region of the protein. The replacement of amino acids can also change the selectivity of binding to cell surface receptors. Ostade et al., Nature 361:266-268 (1993) describes certain mutations resulting in selective binding of TNF-alpha to only one of the two known types of TNF receptors. Thus, the polypeptides of the present invention may include one or more amino acid substitutions, deletions or additions, either from natural mutations or human manipulation.

The invention further includes variations of the BRCC-1 polypeptide which show comparable expression patterns or which include antigenic regions. Such mutants include deletions, insertions, inversions, repeats, and type substitutions. Guidance concerning which amino acid changes are likely to be phenotypically silent can be found in Bowie, J. U., et al., “Deciphering the Message in Protein Sequences: Tolerance to Amino Acid Substitutions,” Science 247:1306-1310(1990).

Of particular interest are substitutions of charged amino acids with another charged amino acid and with neutral or negatively charged amino acids. The latter results in proteins with reduced positive charge to improve the characteristics of the disclosed protein. The prevention of aggregation is highly desirable. Aggregation of proteins not only results in a loss of activity but can also be problematic when preparing pharmaceutical formulations, because they can be immunogenic. (Pinckard et al., Clin. Exp. Immunol. 2:331-340 (1967); Robbins et al., Diabetes 36:838-845 (1987); Cleland et al., Crit. Rev. Therapeutic Drug Carrier Systems 10:307-377 (1993)).

Amino acids in the polypeptides of the present invention that are essential for function can be identified by methods known in the art, such as site-directed mutagenesis or alanine-scanning mutagenesis (Cunningham and Wells, Science 244:1081-1085 (1989)). The latter procedure introduces single alanine mutations at every residue in the molecule. The resulting mutant molecules are then tested for biological activity such as binding to a natural or synthetic binding partner. Sites that are critical for ligand-receptor binding can also be determined by structural analysis such as crystallization, nuclear magnetic resonance or photoaffinity labeling (Smith et al., J Mol. Biol. 224:899-904 (1992) and de Vos et al. Science 255:306-3 12 (1992)). As indicated, changes are preferably of a minor nature, such as conservative amino acid substitutions that do not significantly affect the folding or activity of the protein. Of course, the number of amino acid substitutions a skilled artisan would make depends on many factors, including those described above. Generally speaking, the number of substitutions for any given polypeptide will not be more than 50, 40, 30, 25, 20, 15, 10, 5 or 3.

Thus, in one embodiment, the invention provides a polypeptide comprising (or at least 95% identical to) amino acids from about 1 to about 982 of the amino acid sequence contained in FIG. 6 (SEQ ID NO:3), such as comprising (or at least 95% identical to) amino acids from about 2 to about 982 of the amino acid sequence contained in FIG. 6 (SEQ ID NO:3). In another embodiment, the invention provides a polypeptide wherein, except for at least one conservative amino acid substitution, the polypeptide has amino acids from about 1 to about 982 of the amino acid sequence contained in FIG. 6 (SEQ ID NO:3), such as amino acids from about 2 to about 982 of the open amino acid sequence contained in FIG. 6 (SEQ ID NO:3). In yet another embodiment, the polypeptide can consist essentially of such amino acid sequences. For generating antibodies against the BRCC-1 protein, the invention provides polypeptide comprising an epitope-bearing portion of BRCC-1. The polypeptide comprising an epitope-bearing portion of BRCC-1 can comprise substantially all of the BRCC-1 sequence, but typically comprises a shorter portion of the sequence. Desirably, the polypeptide comprising an epitope-bearing portion of BRCC-1comprises from about 5 to about 30 contiguous amino acids of the protein in FIG. 6, (SEQ ID NO:3) such as from about 10 to about 15 contiguous amino acids of the protein in FIG. 6 (SEQ ID NO:3).

Fusion proteins comprising proteins or polypeptide fragments of BRCC-1 can also be constructed. Fusion proteins are useful for generating antibodies against BRCC-1 amino acid sequences and for use in various assay systems. For example, fusion proteins can be used to identify proteins which interact with a protein of the invention or which interfere with its biological function. Physical methods, such as protein affinity chromatography, or library-based assays for protein-protein interactions, such as the yeast two-hybrid or phage display systems, can also be used for this purpose. Such methods are well known in the art and can also be used as drug screens. Fusion proteins comprising a signal sequence and/or a transmembrane domain of BRCC-1 or a fragment thereof can be used to target other protein domains to cellular locations in which the domains are not normally found, such as bound to a cellular membrane or secreted extracellularly.

A fusion protein comprises two protein segments fused together by means of a peptide bond. Amino acid sequences for use in fusion proteins of the invention can utilize the amino acid sequence shown in FIG. 6 (SEQ ID NO 3) or can be prepared from biologically active variants of FIG. 6 (SEQ ID NO 3), such as those described above. The first protein segment can consist of a full-length BRCC-1.

Other first protein segments can consist of at least 8, 10, 12, 15, 18, 19, 20, 25, 50, 75, 100, 200 contiguous amino acids selected from SEQ ID NO 3. The contiguous amino acids listed herein are not limiting and also include all intermediate lengths such as 20, 21, 22, etc.; 70, 71, 72, etc.

The second protein segment can be a full-length protein or a polypeptide fragment. Proteins commonly used in fusion protein construction include R>-galactosidase, li-glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags can be used in fusion protein constructions, including histidine (His) tags, FLAG tags, influenza hemagglutinin (HA) tags, Myc tags, VSV-G tags, and thioredoxin (Trx) tags. Other fusion constructions can include maltose binding protein (MBP), S-tag, Lex a DNA binding domain (DBD) fusions, GAL4 DNA binding domain fusions, and herpes simplex virus (HSV) BP 16 protein fusions.

These fusions can be made, for example, by covalently linking two protein segments or by standard procedures in the art of molecular biology. Recombinant DNA methods can be used to prepare fusion proteins, for example, by making a DNA construct which comprises a coding sequence contained in FIG. 5 (SEQ ID NO 2) in proper reading frame with a nucleotide encoding the second protein segment and expressing the DNA construct in a host cell, as is known in the art. Many kits for constructing fusion proteins are available from companies that supply research labs with tools for experiments, including, for example, Promega Corporation (Madison, Wis.), Stratagene (La Jolla, Calif.), Clontech (Mountain View, Calif.), Santa Cruz Biotechnology (Santa Cruz, Calif.), MBL International Corporation (MIC; Watertown, Mass.), and Quantum Biotechnologies (Montreal, Canada; 1-888-DNA-KITS).

Proteins, fusion proteins, or polypeptides of the invention can be produced by recombinant DNA methods. For production of recombinant proteins, fusion proteins, or polypeptides, a coding sequence of the nucleotide sequence shown in FIG. 5 (SEQ ID NO 2) can be expressed in prokaryotic or eukaryotic host cells using expression systems known in the art. These expression systems include bacterial, yeast, insect, and mammalian cells. The resulting expressed protein can then be purified from the culture medium or from extracts of the cultured cells using purification procedures known in the art. For example, for proteins fully secreted into the culture medium, cell-free medium can be diluted with sodium acetate and contacted with a cation exchange resin, followed by hydrophobic interaction chromatography. Using this method, the desired protein or polypeptide is typically greater than 95% pure. Further purification can be undertaken, using, for example, any of the techniques listed above.

It may be desireable to modify a protein produced in yeast or bacteria, for example by phosphorylation or glycosylation of the appropriate sites, in order to obtain a functional protein. Such covalent attachments can be made using known chemical or enzymatic methods.

BRCC-1 can also include hybrid and modified forms of BRCC-1 proteins including fusion proteins, BRCC-1 fragments and hybrid and modified forms in which certain amino acids have been deleted or replaced, modifications such as where one or more amino acids have been changed to a modified amino acid or unusual amino acid, and modifications such as glycosylations so long as the hybrid or modified form retains at least one of the biological activities of BRCC-1. By retaining the biological activity of BRCC-1, it is meant that the protein modulates cancer cell proliferation or apoptosis, although not necessarily at the same level of potency as that of BRCC-1 as described herein.

Also included within the meaning of substantially homologous is any BRCC-1 which may be isolated by virtue of cross-reactivity with antibodies to the BRCC-1 described herein or whose encoding nucleotide sequences including genomic DNA, mRNA or cDNA may be isolated through hybridization with the complementary sequence of genomic or subgenomic nucleotide sequences or cDNA of the BRCC-1 herein or fragments thereof. It will also be appreciated by one skilled in the art that degenerate DNA sequences can encode human BRCC-1 and these are also intended to be included within the present invention as are allelic variants of BRCC-1.

Preferred BRCC-1 of the present invention have been identified and isolated in purified form as described. Also preferred is BRCC-1 prepared by recombinant DNA technology. By “pure form” or “purified form” or “substantially purified form” it is meant that a BRCC-1 composition is substantially free of other proteins which are not BRCC-1.

The present invention also includes therapeutic or pharmaceutical compositions comprising BRCC-1 in an effective amount for treating patients with disease, and a method comprising administering a therapeutically effective amount of BRCC-1. These compositions and methods are useful for treating a number of diseases including cancer. One skilled in the art can readily use a variety of assays known in the art to determine whether BRCC-1 would be useful in promoting survival or functioning in a particular cell type.

BRCC-1 protein or polypeptide of the invention can also be expressed in cultured host cells in a form which will facilitate purification. For example, a protein or polypeptide can be expressed as a fusion protein comprising, for example, maltose binding protein, glutathione-S-transferase, orthioredoxin, and purified using a commercially available kit. Kits for expression and purification of such fusion proteins are available from companies such as New England BioLabs, Pharmacia, and Invitrogen. Proteins, fusion proteins, or polypeptides can also be tagged with an epitope, such as a “Flag” epitope (Kodak), and purified using an antibody which specifically binds to that epitope.

The coding sequence disclosed herein can also be used to construct transgenic animals, such as cows, goats, pigs, or sheep. Female transgenic animals can then produce proteins, polypeptides, or fusion proteins of the invention in their milk. Methods for constructing such animals are known and widely used in the art.

Alternatively, synthetic chemical methods, such as solid phase peptide synthesis, can be used to synthesize a secreted protein or polypeptide. General means for the production of peptides, analogs or derivatives are outlined in Chemistry and Biochemistry of Amino Acids, Peptides, and Proteins—A Survey of Recent Developments, B. Weinstein, ed. (1983). Substitution of D-amino acids for the normal L-stereoisomer can be carried out to increase the half-life of the molecule.

The invention further provides polynucleotide constructs encoding BRCC-1 and fragments thereof (such as those described herein), as well as polynucleotides hybridizing to, and antisense to such sequences. For example, in one embodiment, the invention provides a nucleic acid molecule encoding amino acids from about 1 to about 982 of the amino acid sequence contained in FIG. 6 (SEQ IDNO:3), such as encoding amino acids from about 2 to about 982 of the amino acid sequence contained in FIG. 6 (SEQ IDNO:3). In another embodiment, the inventive polynucleotide can be the complement of a nucleic acid molecule encoding amino acids from about 1 to about 982 of the amino acid sequence contained in FIG. 6 (SEQ IDNO:3), such as encoding amino acids from about 2 to about 982 of the amino acid sequence contained in FIG. 6 (SEQ ID NO:3). Of course, the polynucleotide need not be identical, or exactly complementary to such a coding polynucleotide. Thus, for example, the polypeptide can encode (or be substantially complementary to a polynucleotide encoding) a polypeptide wherein, except for at least one conservative amino acid substitution, the polypeptide has amino acids from about 1 to about 982 of the amino acid sequence contained in FIG. 6 (SEQ IDNO:3), such as amino acids from about 2 to about 982 of the open amino acid sequence contained in FIG. 6 (SEQ ID NO:3). In yet another embodiment, the encoded polypeptide can consist essentially of such amino acid sequences. Alternatively, the polynucleotide of the invention can be or comprise a sequence at least about 90% identical to the coding or complementary polynucleotides (such as at least about 95% identical or even at least about 98% identical to such polynucleotides).

In another embodiment, the invention provides a nucleic acid molecule comprising or consisting essentially of about 3 to about 3722 contiguous nucleotides from the nucleic acid sequence identified in FIG. 5 (SEQ ID NO:2), and more preferably comprising or consisting essentially of from about 50 to about 300 (e.g., comprising or consisting essentially of from about 40 to about 250 contiguous nucleotides) from the nucleic acid sequence identified in FIG. 5 (SEQ IDNO:2). Preferably, the nucleic acid molecule comprises or consists essentially of from about 2 to about 324 contiguous nucleotides of the nucleic acid sequence contained in FIG. 5 (SEQ ID NO:2). The polynucleotides and nucleic acid molecules of the invention can be of any desdired type (e.g., DNA, RNA, etc.); however, preferably the nucleic acid molecule is DNA.

Polynucleotide molecules comprising the coding sequences disclosed herein can be used in a polynucleotide construct (or recombinant vector), such as a DNA or RNA construct. Polynucleotide molecules of the invention can be used, for example, in an expression construct to express all or a portion of a protein, variant, fusion protein, or single-chain antibody in a host cell. An expression construct comprises a promoter, which is functional in a chosen host cell. The vector is constructed by inserting the inventive nucleic acid molecule into the vector in operable linkage with the promoter. The skilled artisan can readily select an appropriate promoter from the large number of cell type-specific promoters known and used in the art. The expression construct can also contain a transcription terminator which is functional in the host cell. The expression construct comprises a polynucleotide segment which encodes all or a portion of the desired protein. The polynucleotide segment is located downstream from the promoter. Transcription of the polynucleotide segment initiates at the promoter. The expression construct can be linear or circular and can contain sequences, if desired, for autonomous replication. However constructed, the invention also provides a recombinant vector (e.g., plasmid, viral, etc.) comprising a promoter in operable linkage with a BRCC-1 polynucleotide as described herein (e.g., a BRCC1-coding polynucleotide, complementary polynucleotide, or antisense polynucleotide).

An expression construct can be introduced into a host cell. The host cell comprising the expression construct can be any suitable prokaryotic or eukaryotic cell. Expression systems in bacteria include those described in Chang et al., Nature (1978) 275:615; Goeddel et al., Nature (1979) 281: 544; Goeddel et al., Nucleic Acids Res. (1980) 8:4057; EP 36,776; U.S. Pat. No. 4,551,433; deBoer et al., Proc. Natl. Acad Sci. USA (1983) 80: 21-25; and Siebenlist et al., Cell (1980) 20: 269.

Expression systems in yeast include those described in Hinnnen et al., Proc. Natl. Acad. Sci. USA (1978) 75: 1929; Ito et al., J Bacteriol. (1983) 153: 163; Kurtz et al., Mol. Cell. Biol. (1986) 6:142; Kunze et al., J Basic Microbiol. (1985) 25: 141; Gleeson et al., J. Gen. Microbiol. (1986) 132: 3459, Roggenkamp et al., Mol. Gen. Genet. (1986) 202:302); Das et al., J Bacteriol. (1984) 158: 1165; De Louvencourt et al., J Bacteriol. (1983) 154: 737, Van den Berg et al., Bio/Technology (1990)8: 135; Kunze et al., J. Basic Microbiol. (1985)25: 141; Gregg et al., Mol. Cell. Biol. (1985) 5: 3376; U.S. Pat. No. 4,837,148; U.S. 4,929,555; Beach and Nurse, Nature (1981) 300: 706; Davidow et al., Curr. Genet. (1985) 1p: 380; Gaillardin et al., Curr. Genet. (1985) 10: 49; Ballance et al., Biochem. Biophys. Res. Commun. (1983) 112: 284-289; Tilbum et al., Gene (1983) 26: 205-22; Yelton et al., Proc. Natl. Acad, Sci. USA (1984) 81: 1470-1474; Kelly and Hynes, EMBO J. (1985) 4: 475-479; EP 244,234; and WO 91/00000.

Expression of heterologous genes in insects can be accomplished as described in U.S. Pat. No. 4,745,051; Friesen et al. (1986) “The Regulation of Baculovirus Gene Expression” in: THE MOLECULAR BIOLOGY OF BACULOVIRUSES (W. Doerfler, ed.); EP 127,839; EP 155,476; Vlak et al., J. Gen. Virol. (1988) 69: 765-776; Miller et al., Ann. Rev. Microbiol. (1988) 42: 177; Carbonell et al., Gene (1988) 73: 409; Maeda et al., Nature (1985) 315: 592-594; Lebacq-Verheyden et al., Mol. Cell Biol. (1988) 8: 3129; Smith et al., Proc. Natl. Acad. Sci. USA (1985) 82: 8404; Miyajima et al., Gene (1987) 58: 273; and Martin et al., DNA (1988) 7:99. Numerous baculoviral strains and variants and corresponding permissive insect host cells from hosts are described in Luckow et al., Bio/Technology (1988) 6: 47-55, Miller et al., in GENERIC ENGINEERING (Setlow, J. K. et al. eds.), Vol.

(Plenum Publishing, 1986), pp. 277-279; and Maeda et al., Nature, (1985) 315: 592-594.

Mammalian expression can be accomplished as described in Dijkema et al., EMBO J. (1985) 4: 761; Gormanetal., Proc. Natl. Acad. Sci. USA (1982b) 79: 6777; Boshart et al., Cell (1985) 41: 521; and U.S. Pat. No. 4,399,216. Other features of mammalian expression can be facilitated as described in Ham and Wallace, Meth Enz. (1979) 58: 44; Barnes and Sato, Anal. Biochem. (1980) 102: 255; U.S. Pat. No. 4,767,704; U.S. 4,657,866; U.S. 4,927,762; U.S. 4,560,655; WO 90/103430, WO 87/00005, and U.S. Pat. No. RE 30,985.

Expression constructs can be introduced into host cells using any technique known in the art. These techniques include transferrin-polycation-mediated DNA transfer, transfection with naked or encapsulated nucleic acids, liposome-mediated cellular fusion, intracellular transportation of DNA-coated latex beads, protoplast fusion, viral infection, electroporation, “gene gun,” and calcium phosphate-mediated transfection.

Expression of an endogenous gene encoding a protein of the invention can also be manipulated by introducing by homologous recombination a DNA construct comprising a transcription unit in frame with the endogenous gene, to form a homologously recombinant cell comprising the transcription unit. The transcription unit comprises a targeting sequence, a regulatory sequence, an exon, and an unpaired splice donor site. The new transcription unit can be used to turn the endogenous gene on or off as desired. This method of affecting endogenous gene expression is taught in U.S. Pat. No. 5,641,670.

The targeting sequence is a segment of at least 10, 12, 15, 20, or 50 contiguous nucleotides from the nucleotide sequence shown in FIG. 5 (SEQ ID NO 2). The transcription unit is located upstream to a coding sequence of the endogenous gene. The exogenous regulatory sequence directs transcription of the coding sequence of the endogenous gene.

In certain circumstances, it may be desirable to modulate or decrease the amount of BRCC-1 expressed. Thus, in another aspect of the present invention, BRCC-1 anti-sense oligonucleotides can be made and a method utilized for diminishing the level of expression of BRCC-1 by a cell comprising administering one or more BRCC-1 anti-sense oligonucleotides. By BRCC-1 anti-sense oligonucleotides reference is made to oligonucleotides that have a nucleotide sequence that interacts through base pairing with a specific complementary nucleic acid sequence involved in the expression of BRCC-1 such that the expression of BRCC-1 is reduced. Preferably, the specific nucleic acid sequence involved in the expression of BRCC-1 is a genomic DNA molecule or mRNA molecule that encodes BRCC-1. This genomic DNA molecule can comprise regulatory regions of the BRCC-1 gene, or the coding sequence for mature BRCC-1 protein.

The term complementary to a nucleotide sequence in the context of BRCC-1 antisense oligonucleotides and methods therefor means sufficiently complementary to such a sequence as to allow hybridization to that sequence in a cell, i.e., under physiological conditions. The BRCC-1 antisense oligonucleotides preferably comprise a sequence containing from about 8 to about 100 nucleotides and more preferably the BRCC-1 antisense oligonucleotides comprise from about 15 to about 30 nucleotides. The BRCC-1 antisense oligonucleotides can also contain a variety of modifications that confer resistance to nucleolytic degradation such as, for example, modified internucleoside linages (Uhlmann and Peyman, Chemical Reviews 90:543-548 1990; Schneider and Banner, Tetrahedron Lett. 31:335, 1990 which are incorporated by reference), modified nucleic acid bases as disclosed in 5,958,773 and patents disclosed therein, and/or sugars and the like.

Any modifications or variations of the antisense molecule which are known in the art to be broadly applicable to antisense technology are included within the scope of the invention. Such modifications include preparation of phosphorus-containing linkages as disclosed in U.S. Pat. Nos. 5,536,821; 5,541,306; 5,550,111; 5,563,253; 5,571,799; 5,587,361, 5,625,050 and 5,958,773. The antisense compounds of the invention can include modified bases. The antisense oligonucleotides of the invention can also be modified by chemically linking the oligonucleotide to one or more moieties or conjugates to enhance the activity, cellular distribution, or cellular uptake of the antisense oligonucleotide. Such moieties or conjugates include lipids such as cholesterol, cholic acid, thioether, aliphatic chains, phospholipids, polyamines, polyethylene glycol (PEG), palmityl moieties, and others as disclosed in, for example, U.S. Pat. Nos. 5,514,758, 5,565,552, 5,567,810, 5,574,142, 5,585,481, 5,587,371, 5,597,696 and 5,958,773. Chimeric antisense oligonucleotides are also within the scope of the invention, and can be prepared from the present inventive oligonucleotides using the methods described in, for example, U.S. Pat. Nos. 5,013,830, 5,149,797, 5,403,711, 5,491,133, 5,565,350, 5,652,355, 5,700,922 and 5,958,773.

In the antisense art, a certain degree of routine experimentation is required to select optimal antisense molecules for particular targets. To be effective, the antisense molecule preferably is targeted to an accessible, or exposed, portion of the target RNA molecule. Although in some cases information is available about the structure of target mRNA molecules, the current approach to inhibition using antisense is via experimentation. mRNA levels in the cell can be measured routinely in treated and control cells by reverse transcription of the mRNA and assaying the cDNA levels. The biological effect can be determined routinely by measuring cell growth, proliferation or viability as is known in the art. Assays for measuring apoptosis are also known. Measuring the specificity of antisense activity by assaying and analyzing cDNA levels is an art-recognized method of validating antisense results. It has been suggested that RNA from treated and control cells should be reverse-transcribed and the resulting cDNA populations analyzed. (Branch, A. D., T.I.B.S. 23:45-50, 1998.)

The BRCC-1 polynucleotides and polypeptides of the present invention may be utilized in gene delivery vehicles. The gene delivery vehicle may be of viral or non-viral origin (see generally, Jolly, Cancer Gene Therapy 1:51-64 (1994); Kimura, Human Gene Therapy 5:845-852 (1994); Connelly, Human Gene Therapy 1:185-193 (1995); and Kaplitt, Nature Genetics 6:148-153 (1994)). Gene therapy vehicles for delivery of constructs including a coding sequence of a therapeutic of the invention can be administered either locally or systemically. These constructs can utilize viral or non-viral vector approaches. Expression of such coding sequences can be induced using endogenous mammalian or heterologous promoters. Expression of the coding sequence can be either constitutive or regulated.

The present invention can employ recombinant retroviruses which are constructed to carry or express a selected nucleic acid molecule of interest. Retrovirus vectors that can be employed include those described in EP 0 415 731; WO 90/07936; WO 94/03622; WO 93/25698; WO 93/25234; U.S. Pat. No. 5,219,740; WO 93/11230; WO 93/10218; Vile and Hart, Cancer Res. 53:3860-3864 (1993); Vile and Hart, Cancer Res. 53:962-967 (1993); Ram et al., Cancer Res. 53:83-88 (1993); Takamiya et al., J. Neurosci. Res. 33:493-503 (1992); Baba et al., J. Neurosurg. 79:729-735 (1993); U.S. Pat. No. 4,777,127; GB Patent No. 2,200,651; and EP 0 345 242. Preferred recombinant retroviruses include those described in WO 91/02805.

Packaging cell lines suitable for use with the above-described retroviral vector constructs may be readily prepared (see PCT publications WO 95/30763 and WO 92/05266), and used to create producer cell lines (also termed vector cell lines) for the production of recombinant vector particles. Within particularly preferred embodiments of the invention, packaging cell lines are made from human (such as HT1080 cells) or mink parent cell lines, thereby allowing production of recombinant retroviruses that can survive inactivation in human serum.

The present invention also employs alphavirus-based vectors that can function as gene delivery vehicles. Such vectors can be constructed from a wide variety of alphaviruses, including, for example, Sindbis virus vectors, Semliki forest virus (ATCC VR-67; ATCC VR-1247), Ross River virus (ATCC VR-373; ATCC VR-1246) and Venezuelan equine encephalitis virus (ATCC VR-923; ATCC VR-1250; ATCC VR 1249; ATCC VR-532). Representative examples of such vector systems include those described in U.S. Pat. Nos. 5,091,309; 5,217,879; and 5,185,440; and PCT Publication Nos. WO 92/10578; WO 94/21792; WO 95/27069; WO 95/27044; and WO 95/07994.

Gene delivery vehicles of the present invention can also employ parvovirus such as adeno-associated virus (AAV) vectors. Representative examples include the AAV vectors disclosed by Srivastava in WO 93/09239, Samulski et al., J. Vir. 63:3822-3828 (1989); Mendelson et al., Virol. 166:154-165 (1988); and Flotte et al., P.N.A.S. 90:10613-10617 (1993).

Representative examples of adenoviral vectors include those described by Berkner, Biotechniques 6:616-627 (Biotechniques); Rosenfeld et al., Science 252:431-434 (1991); WO 93/19191; Kolls et al., P.N.A.S. 215-219 (1994); Kass-Bisleretal., P.N.A.S. 90:11498-11502 (1993); Guzman etal., Circulation 88:2838-2848 (1993); Guzman etal., Cir. Res. 73:1202-1207 (1993); Zabneretal., Cell 75:207-216 (1993); Li et al., Hum. Gene Ther. 4:403-409 (1993); Cailaud et al., Eur. J. Neurosci. 5:1287-1291 (1993); Vincent et al., Nat. Genet. 5:130-134 (1993); Jaffe et al., Nat. Genet. 1:372-378 (1992); and Levrero et al., Gene 101:195-202 (1992). Exemplary adenoviral gene therapy vectors employable in this invention also include those described in WO 94/12649, WO 93/03769; WO 93/19191; WO 94/28938; WO 95/11984 and WO 95/00055. Administration of DNA linked to killed adenovirus as described in Curiel, Hum. Gene Ther. 3:147-154 (1992) may be employed.

Other gene delivery vehicles and methods may be employed, including polycationic condensed DNA linked or unlinked to killed adenovirus alone, for example Curiel, Hum. Gene Ther. 3:147-154 (1992); ligand-linked DNA, for example see Wu, J. Biol. Chem. 264:16985-16987 (1989); eukaryotic cell delivery vehicles cells; deposition of photopolymerized hydrogel materials; hand-held gene transfer particle gun, as described in U.S. Pat. No. 5,149,655; ionizing radiation as described in U.S. Pat. No. 5,206,152 and in WO 92/11033; nucleic charge neutralization or fusion with cell membranes. Additional approaches are described in Philip, Mol. Cell Biol. 14:2411-2418 (1994), and in Woffendin, Proc. Natl. Acad. Sci. 91:1581-1585 (1994).

Naked DNA may also be employed. Exemplary naked DNA introduction methods are described in WO 90/11092 and U.S. Pat. No. 5,580,859. Uptake efficiency may be improved using biodegradable latex beads. DNA coated latex beads are efficiently transported into cells after endocytosis initiation by the beads. The method may be improved further by treatment of the beads to increase hydrophobicity and thereby facilitate disruption of the endosome and release of the DNA into the cytoplasm. Liposomes that can act as gene delivery vehicles are described in U.S. Pat. No. 5,422,120, PCT Patent Publication Nos. WO 95/13796, WO 94/23697, and WO 9 1/14445, and EP No. 0 524 968.

Further non-viral delivery suitable for use includes mechanical delivery systems such as the approach described in Woffendin et al., Proc. Natl. Acad. Sci. USA 91(24):11581-11585 (1994). Moreover, the coding sequence and the product of expression of such can be delivered through deposition of photopolymerized hydrogel materials. Other conventional methods for gene delivery that can be used for delivery of the coding sequence include, for example, use of hand-held gene transfer particle gun, as described in U.S. Pat. No. 5,149,655; use of ionizing radiation for activating transferred gene, as described in U.S. Pat. No. 5,206,152 and PCT Patent Publication No. WO 92/11033.

The invention provides a method of modulating apoptosis or proliferation of a cancer cell by regulating expression of BRCC-1 in the mammalian cell. Regulation of the expression of BRCC-1 can be accomplished by transforming the cell with a vector (e.g, a plasmid, viral, or naked DNA vector) comprising or encoding, for example, an antisense oligonucleotide corresponding to the BRCC-1 sequence in FIG. 6. Alternatively, a small molecule inhibitor of BRCC-1, a ribozyme or RNAi can be used to inhibit BRCC-1 expression or activity in the cell or cells. The effect of inhibition of BRCC-1 expression in the cells is to modulate the proliferation of cancer cells and it can be employed in vivo to modulate (e.g., inhibit) cancer proliferation and/or metastasis within a patient. In yet another embodiment, the invention provides a method of treating cancer within a patient, especially a cancer characterized by BRCC-1 overexpression, involving administering an antibody that specifically binds BRCC-1 (e.g., as described herein) to the patient. The inhibition of BRCC-1 in accordance with the inventive method also can be accomplished in combination with radiotherapy; chemotherapy, hormone, biological anticancer agent; hormones or inhibitors of cell cycle dependent kinases. While many cancers can be treated in accordance with the inventive method, it is believed particularly suitable for treating breast cancer or lung cancer.

In another embodiment, the invention provides a method of expressing BRCC-1 in a cell comprising transferring to the cell an agent that promotes expression of BRCC-1. The agent can be, for example, an upstream regulator of BRCC-1, a signaling molecule, or other promoter of BRCC-1 expression. Preferably, however, the agent that promoters expression of BRCC-1 comprises a vector encoding BRCC-1 (e.g., BRCC-1 cDNA). The method can be used to produce BRCC-1 in vitro, or in vivo. When used in vivo, the method can be employed therapeutically, e.g., to treat a condition within a patient characterized by BRCC-1 underexpression. In another embodiment, the invention provides a method for treating a condition within a patient characterized by BRCC-1 underexpression that involves administering a BRCC-1 peptide or fragment thereof to the patient.

In another embodiment, the invention provides a method for treating cancer within a patient that involves overexpression of BRCC-1. In accordance with this aspect of the invention, overexpression of BRCC-1 is promoted in the cancerous cells of the patient, which inhibits cancer cell proliferation and/or metastatic potential of the cancer. Desirably, overexpression of BRCC-1 is accomplished by administering a vector encoding BRCC-1, e.g., BRCC-1 cDNA. The invention provides a composition that includes an agent promoting the overexpression of BRCC-1 or its activity and a pharmaceutically acceptable carrier.

The therapeutic or pharmaceutical compositions of the present invention can be administered by any suitable route known in the art including for example intravenous, subcutaneous, intramuscular, transdermal, intrathecal or intracerebral. Administration can be either rapid as by injection or over a period of time as by slow infusion or administration of slow release formulation.

BRCC-1 can also be linked or conjugated with agents that provide desirable pharmaceutical or pharmacodynamic properties. For example, BRCC-1 can be coupled to any substance known in the art to promote penetration or transport across the blood-brain barrier such as an antibody to the transferrin receptor, and administered by intravenous injection (see, for example, Friden et al., Science 259:373-377, 1993 which is incorporated by reference). Furthermore, BRCC-1 can be stably linked to a polymer such as polyethylene glycol to obtain desirable properties of solubility, stability, half-life and other pharmaceutically advantageous properties. (See, for example, Davis et al., Enzyme Eng. 4:169-73, 1978; Buruham, Am. J. Hosp. Pharm. 51:210-218, 1994 which are incorporated by reference.). A particularly preferred formulation for administration of BRCC-1, anibodies thereto, or nucleotides (especially a BRCC-1 antisense oligonucleotide) is a liposomal formulation, which can be prepared by methods known in the art.

The compositions are usually employed in the form of pharmaceutical preparations. Such preparations are made in a manner well known in the pharmaceutical art. One preferred preparation utilizes a vehicle of physiological saline solution, but it is contemplated that other pharmaceutically acceptable carriers such as physiological concentrations of other non-toxic salts, five percent aqueous glucose solution, sterile water or the like may also be used. It may also be desirable that a suitable buffer be present in the composition. Such solutions can, if desired, be lyophilized and stored in a sterile ampoule ready for reconstitution by the addition of sterile water for ready injection. The primary solvent can be aqueous or alternatively non-aqueous. BRCC-1 can also be incorporated into a solid or semi-solid biologically compatible matrix which can be implanted into tissues requiring treatment.

The carrier can also contain other pharmaceutically-acceptable excipients for modifying or maintaining the pH, osmolarity, viscosity, clarity, color, sterility, stability, rate of dissolution, or odor of the formulation. Similarly, the carrier may contain still other pharmaceutically-acceptable excipients for modifying or maintaining release or absorption or penetration across the blood-brain barrier. Such excipients are those substances usually and customarily employed to formulate dosages for parenteral administration in either unit dosage or multi-dose form or for direct infusion into the cerebrospinal fluid by continuous or periodic infusion.

Dose administration can be repeated depending upon the pharmacokinetic parameters of the dosage formulation and the route of administration used.

It is also contemplated that certain formulations containing BRCC-1 are to be administered orally. Such formulations are preferably encapsulated and formulated with suitable carriers in solid dosage forms. Some examples of suitable carriers, excipients, and diluents include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, and the like. The formulations can additionally include lubricating agents, wetting agents, emulsifying and suspending agents, preserving agents, sweetening agents or flavoring agents. The compositions may be formulated so as to provide rapid, sustained, or delayed release of the active ingredients after administration to the patient by employing procedures well known in the art. The formulations can also contain substances that diminish proteolytic degradation and promote absorption such as, for example, surface active agents.

The specific dose is calculated according to the approximate body weight or body surface area of the patient or the volume of body space to be occupied. The dose will also be calculated dependent upon the particular route of administration selected. Further refinement of the calculations necessary to determine the appropriate dosage for treatment is routinely made by those of ordinary skill in the art. Such calculations can be made without undue experimentation by one skilled in the art in light of the activity disclosed herein in assay preparations of target cells. Exact dosages are determined in conjunction with standard dose-response studies. It will be understood that the amount of the composition actually administered will be determined by a practitioner, in the light of the relevant circumstances including the condition or conditions to be treated, the choice of composition to be administered, the age, weight, and response of the individual patient, the severity of the patient's symptoms, and the chosen route of administration.

In one embodiment of this invention, BRCC-1 may be therapeutically administered by implanting into patients vectors or cells capable of producing a biologically-active form of BRCC-1 or a precursor of BRCC-1, i.e., a molecule that can be readily converted to a biological-active form of BRCC-1 by the body. In one approach cells that secrete BRCC-1 may be encapsulated into semipermeable membranes for implantation into a patient. The cells can be cells that normally express BRCC-1 or a precursor thereof or the cells can be transformed to express BRCC-1 or a precursor thereof. It is preferred that the cell be of human origin and that the BRCC-1 be human BRCC-1 when the patient is human. However, the formulations and methods herein can be used for veterinary as well as human applications and the term “patient” as used herein is intended to include human and veterinary patients.

In a number of circumstances it would be desirable to determine the levels of BRCC-1 in a patient. The identification of BRCC-1 along with the present report showing expression of BRCC-1 provides the basis for the conclusion that the presence of BRCC-1 serves a normal physiological function related to cell growth and survival. Endogenously produced BRCC-1 may also play a role in certain disease conditions, notably cancer. In this respect, the invention provides a method of detecting cancer characterized by BRCC-1 overexpression or BRCC-1 underexpression. In accordance with the method, the levels of BRCC-1 expression in a tissue is detected and then correlated to the presence or absence of cancer. The term “detection” as used herein in the context of detecting the presence of BRCC-1 in a patient is intended to include the determining of the amount of BRCC-1 or the ability to express an amount of BRCC-1 in tissue of a patient, the estimation of prognosis in terms of probable outcome of a disease and prospect for recovery, the monitoring of the BRCC-1 levels over a period of time as a measure of status of the condition, and the monitoring of BRCC-1 levels for determining a preferred therapeutic regimen for the patient. In accordance with the inventive method, detection can be effected by using a cDNA that hybridizes BRCC-1 mRNA (e.g., a probe), using an antibody that specifically binds BRCC-1, or by other suitable methodology.

To detect the presence of BRCC-1 in a patient, a tissue sample is obtained from the patient. The sample can be a tissue or tumor biopsy sample or a sample of blood, plasma, serum, CSF or the like. Samples for detecting BRCC-1 can be taken from these tissue. When assessing peripheral levels of BRCC-1, it is preferred that the sample be a sample of blood, plasma or serum. When assessing the levels of BRCC-1 in the central nervous system a preferred sample is a sample obtained from cerebrospinal fluid or neural tissue.

In some instances it is desirable to determine whether the BRCC-1 gene is intact in the patient or in a tissue or cell line within the patient. By an intact BRCC-1 gene, it is meant that there are no alterations in the gene such as point mutations, deletions, insertions, chromosomal breakage, chromosomal rearrangements and the like wherein such alteration might alter production of BRCC-1 or alter its biological activity, stability or the like to lead to disease processes. Thus, in one embodiment of the present invention a method is provided for detecting and characterizing any alterations in the BRCC-1 gene. The method comprises providing an oligonucleotide that contains the BRCC-1 cDNA, genomic DNA or a fragment thereof or a derivative thereof. By a derivative of an oligonucleotide, it is meant that the derived oligonucleotide is substantially the same as the sequence from which it is derived in that the derived sequence has sufficient sequence complementarily to the sequence from which it is derived to hybridize to the BRCC-1 gene. The derived nucleotide sequence is not necessarily physically derived from the nucleotide sequence, but may be generated in any manner including for example, chemical synthesis or DNA replication or reverse transcription or transcription.

Typically, patient genomic DNA is isolated from a cell sample from the patient and digested with one or more restriction endonucleases such as, for example, Taql and Alul. Using the Southern blot protocol, which is well known in the art, this assay determines whether a patient or a particular tissue in a patient has an intact BRCC-1 gene or a BRCC-1 gene abnormality.

Hybridization to a BRCC-1 gene would involve denaturing the chromosomal DNA to obtain a single-stranded DNA; contacting the single-stranded DNA with a gene probe associated with the BRCC-1 gene sequence; and identifying the hybridized DNA-probe to detect chromosomal DNA containing at least a portion of a human BRCC-1 gene.

The term “probe” as used herein refers to a structure comprised of a polynucleotide that forms a hybrid structure with a target sequence, due to complementarity of probe sequence with a sequence in the target region. Oligomers suitable for use as probes may contain a minimum of about 8-12 contiguous nucleotides which are complementary to the targeted sequence and preferably a minimum of about 20.

The BRCC-1 gene probes of the present invention can be DNA or RNA oligonucleotides and can be made by any method known in the art such as, for example, excision, transcription or chemical synthesis. Probes may be labeled with any detectable label known in the art such as, for example, radioactive or fluorescent labels or enzymatic marker. Labeling of the probe can be accomplished by any method known in the art such as by PCR, random priming, end labeling, nick translation or the like. One skilled in the art will also recognize that other methods not employing a labeled probe can be used to determine the hybridization. Examples of methods that can be used for detecting hybridization include Southern blotting, fluorescence in situ hybridization, and single-strand conformation polymorphism with PCR amplification.

Hybridization is typically carried out at 25°-45° C., more preferably at 32°-40° C. and more preferably at 37°-38° C. The time required for hybridization is from about 0.25 to about 96 hours, more preferably from about one to about 72 hours, and most preferably from about 4 to about 24 hours.

BRCC-1 gene abnormalities can also be detected by using the PCR method and primers that flank or lie within the BRCC-1 gene. The PCR method is well known in the art. Briefly, this method is performed using two oligonucleotide primers which are capable of hybridizing to the nucleic acid sequences flanking a target sequence that lies within a BRCC-1 gene and amplifying the target sequence. The terms “oligonucleotide primer” as used herein refers to a short strand of DNA or RNA ranging in length from about 8 to about 30 bases. The upstream and downstream primers are typically from about 20 to about 30 base pairs in length and hybridize to the flanking regions for replication of the nucleotide sequence. The polymerization is catalyzed by a DNA-polymerase in the presence of deoxynucleotide triphosphates or nucleotide analogs to produce double-stranded DNA molecules. The double strands are then separated by any denaturing method including physical, chemical or enzymatic. Commonly, a method of physical denaturation is used involving heating the nucleic acid, typically to temperatures from about 80° C. to 105° C. for times ranging from about 1 to about 10 minutes. The process is repeated for the desired number of cycles.

The primers are selected to be substantially complementary to the strand of DNA being amplified. Therefore, the primers need not reflect the exact sequence of the template, but must be sufficiently complementary to selectively hybridize with the strand being amplified. After PCR amplification, the DNA sequence comprising BRCC-1 or a fragment thereof is then directly sequenced and analyzed by comparison of the sequence with the sequences disclosed herein to identify alterations which might change activity or expression levels or the like.

In another embodiment, a method for detecting BRCC-1 is provided based upon an analysis of tissue expressing the BRCC-1 gene. Certain tissues have been found to express the BRCC-1 gene. The method comprises hybridizing a polynucleotide to mRNA from a sample of tissue that normally expresses the BRCC-1 gene. The sample is obtained from a patient suspected of having an abnormality in the BRCC-1 gene or in the BRCC-1 gene of particular cells.

To detect the presence of mRNA encoding BRCC-1 protein, a sample is obtained from a patient. The sample can be from blood or from a tissue biopsy sample. The sample may be treated to extract the nucleic acids contained therein. The resulting nucleic acid from the sample is subjected to gel electrophoresis or other size separation techniques. The mRNA of the sample is contacted with a DNA sequence serving as a probe to form hybrid duplexes. The use of a labeled probes as discussed above allows detection of the resulting duplex.

When using the cDNA encoding BRCC-1 protein or a derivative of the cDNA as a probe, high stringency conditions can be used in order to prevent false positives, that is the hybridization and apparent detection of BRCC-1 nucleotide sequences when in fact an intact and functioning BRCC-1 gene is not present. When using sequences derived from the BRCC-1 cDNA, less stringent conditions could be used, however, this would be a less preferred approach because of the likelihood of false positives. The stringency of hybridization is determined by a number of factors during hybridization and during the washing procedure, including temperature, ionic strength, length of time and concentration of formamide. These factors are outlined in, for example, Sambrook et al. (Sambrook et al., 1989, supra).

In order to increase the sensitivity of the detection in a sample of mRNA encoding the BRCC-1 protein, the technique of reverse transcription/polymerization chain reaction (RT/PCR) can be used to amplify cDNA transcribed from mRNA encoding the BRCC-1 protein. The method of RT/PCR is well known in the art, and can be performed as follows. Total cellular RNA is isolated by, for example, the standard guanidium isothiocyanate method and the total RNA is reverse transcribed. The reverse transcription method involves synthesis of DNA on a template of RNA using a reverse transcriptase enzyme and a 3′ end primer. Typically, the primer contains an oligo(dT) sequence. The cDNA thus produced is then amplified using the PCR method and BRCC-1 specific primers. (Belyavsky et al., Nucl. Acid Res. 17:2919-2932, 1989; Krug and Berger, Methods in Enzymology, 152:316-325, Academic Press, NY, 1987 which are incorporated by reference). The polymerase chain reaction method is performed as described above using two oligonucleotide primers that are substantially complementary to the two flanking regions of the DNA segment to be amplified. Following amplification, the PCR product is then electrophoresed and detected by ethidium bromide staining or by phosphoimaging.

The present invention further provides for methods to detect the presence and, in some applications, amount of the BRCC-1 protein in a sample obtained from a patient. Any method known in the art for detecting proteins can be used. Such methods include, but are not limited to immunodiffusion, immunoelectrophoresis, immunochemical methods, binder-ligand assays, immunohistochemical techniques, agglutination and complement assays. (Basic and Clinical Immunology, 217-262, Sites and Terr, eds., Appleton & Lange, Norwalk, Conn., 1991, which is incorporated by reference). Preferred are binder-ligand immunoassay methods including reacting antibodies with an epitope or epitopes of the BRCC-1 protein and competitively displacing a labeled BRCC-1 protein or derivative thereof. In such methods, monoclonal or polyclonal antibodies that specifically bind BRCC-1, such as herein described, can be employed.

As used herein, a derivative of the BRCC-1 protein is intended to include a polypeptide in which certain amino acids have been deleted or replaced or changed to modified or unusual amino acids wherein the BRCC-1 derivative is biologically equivalent to BRCC-1 and wherein the polypeptide derivative cross-reacts with antibodies raised against the BRCC-1 protein. By cross-reaction it is meant that an antibody reacts with an antigen other than the one that induced its formation.

Numerous competitive and non-competitive protein binding immunoassays are well known in the art. Antibodies employed in such assays may be unlabeled, for example as used in agglutination tests, or labeled for use in a wide variety of assay methods. Labels that can be used include radionuclides, enzymes, fluorescers, chemiluminescers, enzyme substrates or co-factors, enzyme inhibitors, particles, dyes and the like for use in radioimmunoassay (RIA), enzyme immunoassays, e.g., enzyme-linked immunosorbent assay (ELISA), fluorescent immunoassays and the like.

The invention further provides an antibody (e.g., an isolated antibody) or antiserum containing antibodies that bind(s) specifically to the BRCC-1 peptide. The antibody preferably specifically binds to the natural BRCC-1 polypeptide (SEQ ID NO:3), but also can bind to the BRCC-1 varient peptides as described herein. Polyclonal or monoclonal antibodies to the protein or an epitope thereof can be made for use in immunoassays by any of a number of methods known in the art. By epitope reference is made to an antigenic determinant of a polypeptide. An epitope could comprise 3 amino acids in a spatial conformation which is unique to the epitope. Generally an epitope consists of at least 5 such amino acids. Methods of determining the spatial conformation of amino acids are known in the art, and include, for example, x-ray crystallography and 2 dimensional nuclear magnetic resonance.

One approach for preparing antibodies to a protein is the selection and preparation of an amino acid sequence of all or part of the protein, chemically synthesizing the sequence and injecting it into an appropriate animal, usually a rabbit or a mouse.

Oligopeptides can be selected as candidates for the production of an antibody to the BRCC-1 protein based upon the oligopeptides lying in hydrophilic regions, which are thus likely to be exposed in the mature protein. Peptide sequence used to generate antibodies against any fragment of BRCC-1 that typically is at least 5-6 amino acids in length, optionally fused to an immunogenic carrier protein, e.g. KLH or BSA. Additional oligopeptides can be determined using, for example, the Antigenicity Index, Welling, G. W. et al., FEBS Lett. 188:215-218 (1985), incorporated herein by reference.

In other embodiments of the present invention, humanized monoclonal antibodies are provided, wherein the antibodies are specific for BRCC-1. The phrase “humanized antibody” refers to an antibody derived from a non-human antibody, typically a mouse monoclonal antibody. Alternatively, a humanized antibody may be derived from a chimeric antibody that retains or substantially retains the antigen-binding properties of the parental, non-human, antibody but which exhibits diminished immunogenicity as compared to the parental antibody when administered to humans. The phrase “chimeric antibody,” as used herein, refers to an antibody containing sequence derived from two different antibodies (see, e.g., U.S. Pat. No. 4,816,567) which typically originate from different species. Most typically, chimeric antibodies comprise human and murine antibody fragments, generally human constant and mouse variable regions.

Because humanized antibodies are far less immunogenic in humans than the parental mouse monoclonal antibodies, they can be used for the treatment of humans with far less risk of anaphylaxis. Thus, these antibodies may be preferred in therapeutic applications that involve in vivo administration to a human such as, e.g., use as radiation sensitizers for the treatment of neoplastic disease or use in methods to reduce the side effects of, e.g., cancer therapy.

Humanized antibodies may be achieved by a variety of methods including, for example: (1) grafting the non-human complementarity determining regions (CDRs) onto a human framework and constant region (a process referred to in the art as “humanizing”), or, alternatively, (2) transplanting the entire non-human variable domains, but “cloaking” them with a human-like surface by replacement of surface residues (a process referred to in the art as “veneering”). In the present invention, humanized antibodies will include both “humanized” and “veneered” antibodies. These methods are disclosed in, e.g., Jones et al., Nature 321:522-525 (1986); Morrison et al., Proc. Natl. Acad. Sci, USA, 81:6851-6855 (1984); Morrison and Oi, Adv. Immunol., 44:65-92 (1988); Verhoeyer et al., Science 239:1534-1536 (1988); Padlan, Molec. Immun. 28:489-498 (1991); Radian, Molec. Immunol. 31(3): 169-217 (1994); and Kettleborough, C. A. etal., Protein Eng. 4(7):773-83 (1991) each of which is incorporated herein by reference.

The phrase “complementarity determining region” refers to amino acid sequences which together define the binding affinity and specificity of the natural Fv region of a native immunoglobulin binding site. See, e.g., Chothia et al., J. Mol. Biol. 196:901-917 (1987); Kabat et al., U.S. Dept. of Health and Human Services NIH Publication No. 91-3242 (1991). The phrase “constant region” refers to the portion of the antibody molecule that confers effector functions. In the present invention, mouse constant regions are substituted by human constant regions. The constant regions of the subject humanized antibodies are derived from human immunoglobulins. The heavy chain constant region can be selected from any of the five isotypes: alpha, delta, epsilon, gamma or mu.

One method of humanizing antibodies comprises aligning the non-human heavy and light chain sequences to human heavy and light chain sequences, selecting and replacing the non-human framework with a human framework based on such alignment, molecular modeling to predict the conformation of the humanized sequence and comparing to the conformation of the parent antibody. This process is followed by repeated back mutation of residues in the CDR region which disturb the structure of the CDRs until the predicted conformation of the humanized sequence model closely approximates the conformation of the non-human CDRs of the parent non-human antibody. Such humanized antibodies may be further derivatized to facilitate uptake and clearance, e.g, via Ashwell receptors. See, e.g., U.S. Pat. Nos. 5,530,101 and 5,585,089 which patents are incorporated herein by reference.

Humanized antibodies to BRCC-1 can also be produced using transgenic animals that are engineered to contain human immunoglobulin loci. For example, WO 98/24893 discloses transgenic animals having a human Ig locus wherein the

animals do not produce functional endogenous immunoglobulins due to the inactivation of endogenous heavy and light chain loci. WO 91/10741 also discloses transgenic non-primate mammalian hosts capable of mounting an immune response to an immunogen, wherein the antibodies have primate constant and/or variable regions, and wherein the endogenous immunoglobulin-encoding loci are substituted or inactivated. WO 96/30498 discloses the use of the Cre/Lox system to modify the immunoglobulin locus in a mammal, such as to replace all or a portion of the constant or variable region to form a modified antibody molecule. WO 94/02602 discloses non-human mammalian hosts having inactivated endogenous Ig loci and functional human Ig loci. U.S. Pat. No. 5,939,598 discloses methods of making transgenic mice in which the mice lack endogenous heavy claims, and express an exogenous immunoglobulin locus comprising one or more xenogeneic constant regions.

Using a transgenic animal described above, an immune response can be produced to a selected antigenic molecule, and antibody-producing cells can be removed from the animal and used to produce hybridomas that secrete human monoclonal antibodies. Immunization protocols, adjuvants, and the like are known in the art, and are used in immunization of, for example, a transgenic mouse as described in WO 96/33735. This publication discloses monoclonal antibodies against a variety of antigenic molecules including IL-6, IL-8, TNF, human CD4, L-selectin, gp39, and tetanus toxin. The monoclonal antibodies can be tested for the ability to inhibit or neutralize the biological activity or physiological effect of the corresponding protein. WO 96/33735 discloses that monoclonal antibodies against IL-8, derived from immune cells of transgenic mice immunized with IL-8, blocked IL-8-induced functions of neutrophils. Human monoclonal antibodies with specificity for the antigen used to immunize transgenic animals are also disclosed in WO 96/34096.

In the present invention, BRCC-1 polypeptides of the invention and variants thereof are used to immunize a transgenic animal as described above. Monoclonal antibodies are made using methods known in the art, and the specificity of the antibodies is tested using isolated BRCC-1 polypeptides.

Methods for preparation of the BRCC-1 protein or an epitope thereof include, but are not limited to chemical synthesis, recombinant DNA techniques or isolation from biological samples. Chemical synthesis of a peptide can be performed, for example, by the classical Merrifeld method of solid phase peptide synthesis (Merrifeld, J. Am. Chem. Soc. 85:2149,1963 which is incorporated by reference) or the FMOC strategy on a Rapid Automated Multiple Peptide Synthesis system (E. I. du Pont de Nemours Company, Wilmington, Del.) (Caprino and Han, J. Org. Chem. 37:3404, 1972 which is incorporated by reference).

Polyclonal antibodies can be prepared by immunizing rabbits or other animals by injecting antigen followed by subsequent boosts at appropriate intervals. The animals are bled and sera assayed against purified BRCC-1 protein usually by ELISA or by bioassay based upon the ability to block the action of BRCC-1. In a non-limiting example, an antibody to BRCC-1 can block the binding of BRCC-1 to Dishevelled protein. When using avian species, e.g., chicken, turkey and the like, the antibody can be isolated from the yolk of the egg. Monoclonal antibodies can be prepared after the method of Milstein and Kohler by fusing splenocytes from immunized mice with continuously replicating tumor cells such as myeloma or lymphoma cells. (Milstein and Kohler, Nature 256:495-497, 1975; Gulfre and Milstein, Methods in Enzymology: Immunochemical Techniques 73:1-46, Langone and Banatis eds., Academic Press, 1981 which are incorporated by reference). The hybridoma cells so formed are then cloned by limiting dilution methods and supernates assayed for antibody production by ELISA, RIA or bioassay.

The unique ability of antibodies to recognize and specifically bind to target proteins provides an approach for treating an overexpression of the protein. Thus, another aspect of the present invention provides for a method for preventing or treating diseases involving overexpression of the BRCC-1 protein by treatment of a patient with specific antibodies to the BRCC-1 protein.

Specific antibodies, either polyclonal or monoclonal, to the BRCC-1 protein can be produced by any suitable method known in the art as discussed above. For example, murine or human monoclonal antibodies can be produced by hybridoma technology or, alternatively, the BRCC-1 protein, or an immunologically active fragment thereof, or an anti-idiotypic antibody, or fragment thereof can be administered to an animal to elicit the production of antibodies capable of recognizing and binding to the BRCC-1 protein. Such antibodies can be from any class of antibodies including, but not limited to IgG, IgA, 1gM, IgD, and IgE or in the case of avian species, IgY and from any subclass of antibodies.

The availability of BRCC-1 allows for the identification of small molecules and low molecular weight compounds that inhibit the binding of BRCC-1 to binding partners, through routine application of high-throughput screening methods (HTS). HTS methods generally refer to technologies that permit the rapid assaying of lead compounds for therapeutic potential. HTS techniques employ robotic handling of test materials, detection of positive signals, and interpretation of data. Lead compounds may be identified via the incorporation of radioactivity or through optical assays that rely on absorbance, fluorescence or luminescence as read-outs. Gonzalez, J. E. et al., (1998) Curr. Opin. Biotech. 9:624-631.

Model systems are available that can be adapted for use in high throughput screening for compounds that inhibit the interaction of BRCC-1 with its ligand, for example by competing with BRCC-I for ligand binding. Sarubbi et al., (1996) Anal. Biochem. 237:70-75 describe cell-free, non-isotopic assays for discovering molecules that compete with natural ligands for binding to the active site of IL-I receptor. Martens, C. et al., (1999) Anal. Biochem. 273:20-31 describe a generic particle-based nonradioactive method in which a labeled ligand binds to its receptor immobilized on a particle; label on the particle decreases in the presence of a molecule that competes with the labeled ligand for receptor binding.

BRCC-1 may also be used in screens to identify drugs for treatment of cancers which involve over-activity of the encoded protein, or new targets which would be useful in the identification of new drugs. For all of the preceding embodiments, the clinician will determine, based on the specific condition, whether BRCC-1 polypeptides or polynucleotides (including antisense polynucleotides), antibodies to BRCC-1, or small molecules such as peptide analogues or antagonists, will be the most suitable form of treatment. These forms are all within the scope of the invention.

The present invention has been described with reference to specific embodiments. However, this invention is intended to cover those changes and substitutions, which may be made by those skilled in the art without departing from the spirit and scope of the appended claims.

What is claimed is:

1. An isolated nucleic acid molecule comprising a polynucleotide selected from the group consisting of:
(a) a polynucleotide encoding amino acids from about 1 to about 982 of SEQ ID NO:3;
(b) a polynucleotide encoding amino acids from about 2 to about 982 of SEQ ID NO:3;
(c) the polynucleotide complement of the polynucleotide of (a) or (b); and
(d) a polynucleotide at least 90% identical to the polynucleotide of (a), (b) or (c).

2. An isolated nucleic acid molecule comprising about 3 to about 3722 contiguous nucleotides from SEQ ID NO:2.

3. An isolated nucleic acid molecule comprising about 50 to about 300 contiguous nucleotides from SEQ ID NO:2.

4. An isolated nucleic acid molecule comprising about 2 to about 324 contiguous nucleotides of SEQ ID NO:2.

5. An isolated nucleic acid molecule comprising about 40 to about 250 contiguous nucleotides from SEQ ID NO:2.

6. An isolated nucleic acid molecule comprising a polynucleotide encoding a polypeptide wherein, except for at least one conservative amino acid substitution, said polypeptide has an amino acid sequence selected from the group consisting of:
(a) amino acids from about 1 to about 982 of SEQ ID NO:3; and
(b) amino acids from about 2 to about 982 of SEQ ID NO:3.

7. The isolated nucleic acid molecule of claim 1, which is DNA.

8. A method of making a recombinant vector comprising inserting a nucleic acid molecule of claim 1 into a vector in operable linkage to a promoter.

9. A recombinant vector produced by the method of claim 8.

10. A method of making a recombinant host cell comprising introducing the recombinant vector of claim 9 into a host cell.

11. A recombinant host cell produced by the method of claim 10.

12. A recombinant method of producing a polypeptide, comprising culturing the recombinant host cell of claim 11 under conditions such that said polypeptide is expressed and recovering said polypeptide.

13. An isolated polypeptide comprising amino acids at least 95% identical to amino acids selected from the group consisting of:
(a) amino acids from about 1 to about 982 of SEQ ID NO:3; and
(b) amino acids from about 2 to about 982 of SEQ ID NO:3.

14. An isolated polypeptide wherein, except for at least one conservative amino acid substitution, said polypeptide has an amino acid sequence selected from the group consisting of:
(a) amino acids from about 1 to about 982 of SEQ ID NO:3; and
(b) amino acids from about 2 to about 982 of SEQ ID NO:3.

15. An isolated polypeptide comprising amino acids selected from the group consisting of:
(a) amino acids from about 1 to about 982 of SEQ ID NO:3; and
(b) amino acids from about 2 to 982 of SEQ ID NO:3.

16. An epitope-bearing portion of the polypeptide identified in FIG. 6.

17. The epitope-bearing portion of claim 16, which comprises about 5 to about 30 contiguous amino acids of the protein in FIG. 6.

18. The epitope-bearing portion of claim 17, which comprises about 10 to about 15 contiguous amino acids of the protein in FIG. 6.

19. An isolated antibody that binds specifically to the polypeptide of claim 15.

20. A monoclonal antibody according to claim 19.

21. A method of modulating apoptosis or proliferation of a cancer cell, comprising regulating expression of BRCC-1 in said mammalian cell.

22. The method of claim 21, wherein said mammalian cell is transformed with a vector encoding an antisense oligonucleotide corresponding to the BRCC-1 sequence in FIG. 6.

23. An antisense oligonucleotide that inhibits the expression of BRCC-1 in a mammalian cell.

24. The antisense oligonucleotide of claim 23, which is contained in a liposomal formulation.

25. A method of treating cancer characterized by BRCC-1 overexpression by administration of an antisense oligonucleotide or ribozyme that modulates BRCC-1 expression.

26. A method of treating cancer characterized by BRCC-1 overexpression comprising administering an antibody that specifically binds BRCC-1.

27. A method of detecting cancer characterized by BRCC-1 overexpression or BRCC-1 underexpression comprising detecting the levels of BRCC-1 expression and correlating said level of expression to the presence or absence of cancer.

28. The method of claim 27, which is effected by using a cDNA that hybridizes BRCC-1 and mRNA.

29. The method of claim 27 which is effected by using an antibody that specifically binds BRCC-1.

30. A method for modulating cancer cell proliferation and/or metastasis in a cancer patient comprising administering a ribozyme or antisense oligonucleotide that modulates BRCC-1 expression.

31. The method of claim 30, wherein said cancer is breast cancer or lung cancer.

32. A method of treating a condition characterized by BRCC-1 underexpression comprising administering an agent that promotes BRCC-1 expression.

33. The method of claim 32, wherein administering an agent that promotes BRCC-1 expression comprises administering BRCC-1 cDNA.

34. A method of treating a condition characterized by BRCC-1 underexpression comprising administering an BRCC-1 peptide or a fragment thereof.

35. A method for inhibiting cancer cell proliferation and/or metastasis in a cancer patient comprising administering a small molecule inhibitor of BRCC-1; an antibody that specifically binds BRCC-1; a ribozyme or antisense oligonucleotide that modulates BRCC-1 expression in combination with radiotherapy; chemotherapy, hormone, biological anticancer agent; hormones or inhibitors of cell cycle dependent kinases.

36. A method for treating cancer comprising administering BRCC-1 cDNA that leads to overexpression of BRCC-1, thereby inhibiting cancer cell proliferation and/or metastatic potential.

37. A pharmaceutical composition for the treatment of cancer that comprises an agent promoting the overexpression of BRCC-1 or its activity and a pharmaceutically acceptable carrier.


SEQUENCE LISTING (3 sequences)

SEQ ID NO: 1 - DNA, 483 bp, Artificial
  Nucleotide sequence of partial BRCC1 cDNA clone
aagaggaacatttttcctgtagtatcctcacgagttcttagagtgtcttgaaaaaatatgttggctatgtgaaagaatgcttcaactaaaatggaatgttatgctgttcactcctaaactttgaggagcatcttgatatgttttaacattatcatggcagggaaatatataaagaagaaaaatatttttacattaaaccttttctaaaaattgtaaatagaaaaataatttgattttttatcaagaatgacacttatcaatatatattatgttatattgccaatctgttgagattgactcaaaaggttaaatattgccactgttgaagataattatgagtatcgcaaaccttgtttctgacccattttgatagtttctatatacgcctttaaaatgatgaatgttgcaggttaataaagttaatacctttaaaacttggtgaaataccattacagaagccaaaaataaaaactccctgcctct

SEQ ID NO: 2 - DNA, 3722 bp, Artificial
  Predicted cDNA sequence of BRCC1 gene (GenBank Accession #AF502591, Date of submission 04/15/2002)
aaatatggtctttcttgggcgcgcgcgacaatgtgaggagtggggtggagcgtgtgtggtgtgtggctgcggcctgggcaagagccgccgcggaccatgagctgagtaagttctggagggatcctgcctcttggagccttcgcagccaggcagctgtgaactgtgagctagagtgaagcagaaatctaggaagatgagctccaagatggtcataagtgaaccaggactgaattgggatatttcccccaaaaatggccttaagacatttttctctcgagaaaattataaagatcattccatggctccaagtttaaaagaactacgtgttttatccaacagacgtataggagaaaatttgaatgcctcagcaagttctgtagaaaatgagccggcagttagttcagcaactcaagcaaaggaaaaagttaaaaccacaattggaatggttcttcttccaaaaccaagagttccttatcctcgtttctctcgtttctcacagagagagcagaggagttatgtggacttgttggttaaatacgcaaagattcctgcaaattccaaagctgttggaataaataaaaatgactacttgcagtacttggatatgaaaaaacatgtgaacgaagaagttactgagttcctaaagtttttgcagaattctgcaaagaaatgtgcgcaggattataatatgctttctgatgatgcccgtctcttcacagagaaaattttaagagcttgcattgaacaagtgaaaaagtattcagaattctatactctccacgaggtcaccagcttaatgggattcttcccattcagagtagagatgggattaaagttagaaaaaactcttctcgcattgggcagtgtaaaatatgtgaaaacagtatttccctcaatgcctataaagttgcagctgtcaaaggacgatatagctaccattgaaacgtcagaacaaacagctgaagctatgcattatgatattagtaaagatccaaatgcagagaagcttgtttccagatatcaccctcagatagctctaactagtcagtcattatttaccttattaaataatcatggaccaacgtacaaggaacagtgggaaattccagtgtgtattcaagtaatacctgttgcaggttcaaaaccagttaaagtaatatatattaattcaccacttccccaaaagaaaatgactatgagagagagaaatcaaatctttcatgaagttccattaaaatttatgatgtccaaaaacacatctgttccagtctctgcagtctttatggacaaacctgaagagtttatatctgaaatggacatgtcctgtgaagtcaacgagtgccgaaaaattgagagtcttgaaaacttgtatttggattttgatgatgatgtcacagaacttgaaacttttggagtaaccaccaccaaagtatcaaaatcaccaagtccagcaagtacttccacagtacctaacatgacagatgctcctacagcccccaaagcaggaactacaactgtggcaccaagtgcaccagacatttctgctaattctagaagtttatctcagattctgatggaacaattgcaaaaggagaaacagctggtcactggtatggatggtggccctgaggaatgcaaaaataaagatgatcagggatttgaatcatgtgaaaaggtatcaaattctgacaagcctttgatacaagatagtgacttgaaaacatctgatgccttacagttagaaaattctcaggaaattgaaacttctaataaaaatgatatgactatagatatattacatgctgatggtgaaagacctaatgttctagaaaacctagacaactcaaaggaaaagactgttggatcagaagcagcaaaaactgaagatacagttctctgcagcagtgatacagatgaggagtgtttaatcattgatacagaatgtaaaaataatagtgatggaaagacagctgttgtgggttctaacttaagttccagaccagctagtccaaattcttcctcaggacaggcttctgtaggaaaccagactaatactgcttgtagtcctgaagagtcatgtgttttaaaaaaacctatcaaacgagtatataaaaaatttgatccagttggagagattttaaaaatgcaggatgagctcttaaagccaatttccagaaaagtaccagaattgcccttaatgaatttagaaaattctaaacagccttctgtttctgagcaattgtctggtccttcagactcctctagttggccgaaatctggatggccttctgcatttcagaagccaaaaggacgattgccatatgaacttcaggactatgttgaagatacatcggaatacctagctcctcaggaaggaaattttgtttataagttatttagcctgcaagacctgttgttactcgtacgctgcagtgtccagaggatagagacaagaccacgttctaaaaaacggaagaaaatcagaagacaatttccagtttatgtactaccaaaagtagagtatcaagcttgttacggagttgaagctctgactgaaagtgaactttgtcgcttatggactgaaagtttattgcattccaacagctcattttatgttgggcatatcgatgcatttacttcaaaactttttctactggaagaaattacctcagaagaattaaaagaaaagctttcagcactcaagatttccaatttatttaacatcctccaacacattctaaagaaactaagtagcttgcaggagggttcctacttgttatctcatgcagcagaagattcttcactcctgatttataaggcctctgatggaaaagttactaggacagcatacaatttgtataaaacacattgcggccttcctggtgtaccttccagtctctcagttccctgggtcccattagatcccagcctgttattaccatatcatatccatcatggaagaataccttgtacttttccaccgaaatcactggataccacaacacaacaaaagattggtggaacgagaatgcctacacgcagccacaggaatccagtttccatggaaaccaaaagcagttgcttgcctgctcagcaagttgaaactgaaggagtggctccacataaaagaaaaataacttgaggactgtaccatggaaaactaaatttaaaaaaccagttataacagtgtttaatttagataagtttgagggaaaataatcagtaggcaagaggaacatttttcctgtagtagctagagtgccttgaaaaaatgtgttggctatgtgaaggaatatttcaactaaaatggaatggtatgcttttcacccttaaagtttgaggaggatcttgatatgttttaacattatcatggcagggaaatatataaagaagaaaaatatttttacattaaaccttttctaaaaattgtaaatagaaaaataatttggttttttatcaagaacaacacttatcgttatgtattgtgttagttatattgccagtctgttgcgactgactcaaaaagttaaatgttgccactgctgaagatgattatgagcatcgcaaactttgtttctgacccattttgacagtttttatatactcctttaaaatgatgaatgttacaggttaataaagttaatacctttaaaaacttggtgaaattccattacagaagccaaaaataaaaactccctgcctctgaaaaaaaaaaaaaaaaa

SEQ ID NO: 3 - protein, 982 aa, Artificial
  Predicted amino acid sequence of BRCC1 protein
MSSKMVISEPGLNWDISPKNGLKTFFSRENYKDHSMAPSLKELRVLSNRRIGENLNASASSVENEPAVSSATQAKEKVKTTIGMVLLPKPRVPYPRFSRFSQREQRSYVDLLVKYAKIPANSKAVGINKNDYLQYLDMKKHVNEEVTEFLKFLQNSAKKCAQDYNMLSDDARLFTEKILRACIEQVKKYSEFYTLHEVTSLMGFFPFRVEMGLKLEKTLLALGSVKYVKTVFPSMPIKLQLSKDDIATIETSEQTAEAMHYDISKDPNAEKLVSRYHPQIALTSQSLFTLLNNHGPTYKEQWEIPVCIQVIPVAGSKPVKVIYINSPLPQKKMTMRERNQIFHEVPLKFMMSKNTSVPVSAVFMDKPEEFISEMDMSCEVNECRKIESLENLYLDFDDDVTELETFGVTTTKVSKSPSPASTSTVPNMTDAPTAPKAGTTTVAPSAPDISANSRSLSQILMEQLQKEKQLVTGMDGGPEECKNKDDQGFESCEKVSNSDKPLIQDSDLKTSDALQLENSQEIETSNKNDMTIDILHADGERPNVLENLDNSKEKTVGSEAAKTEDTVLCSSDTDEECLIIDTECKNNSDGKTAVVGSNLSSRPASPNSSSGQASVGNQTNTACSPEESCVLKKPIKRVYKKFDPVGEILKMQDELLKPISRKVPELPLMNLENSKQPSVSEQLSGPSDSSSWPKSGWPSAFQKPKGRLPYELQDYVEDTSEYLAPQEGNFVYKLFSLQDLLLLVRCSVQRIETRPRSKKRKKIRRQFPVYVLPKVEYQACYGVEALTESELCRLWTESLLHSNSSFYVGHIDAFTSKLFLLEEITSEELKEKLSALKISNLFNILQHILKKLSSLQEGSYLLSHAAEDSSLLIYKASDGKVTRTAYNLYKTHCGLPGVPSSLSVPWVPLDPSLLLPYHIHHGRIPCTFPPKSLDTTTQQKIGGTRMPTRSHRNPVSMETKSSCLPAQQVETEGVAPHKRKIT